(19) United States
(12) Patent Application Publication
(10) Pub. No.: US 2026/0325529 A1
(43) Pub. Date: Oct. 22, 2026

(54) CRYSTALLINE 4-CHLORO-N-(4-(MORPHOLINOMETHYL)PHENYL)BENZAMIDE PHOSPHATE SALT AND USES THEREOF

(71) Applicant: GEn1E Lifesciences Inc., Palo Alto, CA (US)

(72) Inventors: Ritu LAL, Palo Alto, CA (US); Nathan Larson, Saint George, UT (US); Adam Galan, Alameda, CA (US)

(21) Appl. No.: 19/642,955

(22) Filed: Apr. 9, 2026

Related U.S. Application Data
(60) Provisional application No. 63/786,127, filed on Apr. 9, 2025.

(51) Int. Cl. A61K 31/5375; C07D 295/135

(52) U.S. Cl. CPC A61K 31/5375; C07D 295/135

(57) ABSTRACT

Crystalline 4-chloro-N-(4-(morpholinomethyl)phenyl)benzamide phosphate salt is disclosed. The crystalline phosphate salts can be used to treat cancer, inflammatory diseases, autoimmune diseases, respiratory diseases, and age-related diseases.

This application claims the benefit under 35 U.S.C. § 119(e) of U.S. Provisional Application No. 63/786,127 filed on Apr. 9, 2025, which is incorporated by reference in its entirety.

### RELATED APPLICATIONS

This application is related to U.S. application Ser. No. 17/231,598, filed on Apr. 15, 2021, which issued as U.S. Pat. No. 11,357,781 B2 on Jun. 14, 2022; U.S. application Ser. No. 17/740,248 filed on May 9, 2022, which issued as U.S. Pat. No. 11,911,392 B2 on Feb. 27, 2024; U.S. application Ser. No. 18/169,785 filed on Feb. 15, 2023, which issued as U.S. Pat. No. 11,911,393 B2 on Feb. 27, 2024; U.S. application Ser. No. 18/416,532 filed on Jan. 18, 2024; U.S. application Ser. No. 18/645,972 filed on Apr. 25, 2024, which issued as U.S. Pat. No. 12,116,353 B1 on Oct. 15, 2024, and U.S. application Ser. No. 18/806,836 filed on Aug. 16, 2024.

### FIELD

The disclosure relates to crystalline 4-chloro-N-(4-(morpholinomethyl)phenyl)benzamide phosphate, pharmaceutical compositions containing crystalline 4-chloro-N-(4-(morpholinomethyl)phenyl)benzamide phosphate, methods of preparing crystalline 4-chloro-N-(4-(morpholinomethyl)phenyl)benzamide phosphate, and methods of treatment using crystalline 4-chloro-N-(4-(morpholinomethyl)phenyl)benzamide phosphate.

### BACKGROUND

Mitogen-activated protein kinases (MAPKs) are serine/threonine protein kinases that process and regulate cellular properties in response to a wide range of extracellular stimuli. These enzymes phosphorylate the hydroxyl group of serine or threonine in proteins and play important roles in the regulation of cell proliferation, differentiation, survival, and apoptosis. In mammalian cells, several distinct MAPKs have been identified, including p38 MAPK.

p38 MAPK is a class of MAPKs responsive to stress stimuli such as inflammatory cytokines and reactive oxygen species (ROS) and is involved in a wide range of signaling pathways that stimulate different biological functions. For example, p38 MAPK plays an essential role in the regulation of pro-inflammatory signaling networks and in the biosynthesis of cytokines, including tumor necrosis factor-α (TNF-α) and interleukin-1β (IL-1β) in immune cells.

Studies have shown that p38 MAPK contributes to the pathogenesis of chronic inflammation, which has led to the identification and development of p38 MAPK inhibitors for treating inflammatory diseases such as rheumatoid arthritis and asthma.

p38 MAPK comprises four isoforms (α, β, γ and δ). p38α MAPK was the first isoform of p38 MAPK to be identified and was first recognized as a stress-induced kinase that can be activated by lipopolysaccharide (LPS) and inflammatory cytokines. Inhibition of p38 MAPK has been shown to effectively alleviate inflammatory diseases such as rheumatoid arthritis, cardiovascular disease, and inflammatory pain.

Many p38 MAPK catalytic inhibitors are poorly effective and cause toxicity, possibly due to activity against non-inflammatory p38 and loss of p38α-dependent counterregulatory responses. p38α MAPK inhibitors that can selectively block certain p38α MAPK functions and preserve critical counterregulatory and homeostatic functions with application for the treatment of inflammatory and oncologic diseases are desired.

4-Chloro-N-(4-(morpholinomethyl)phenyl)benzamide and pharmaceutically acceptable salts are disclosed in U.S. Application Publication No. 2020/0331874 A1 with compounds exhibiting p38α MAPK inhibitory activity.

### SUMMARY

According to the present invention, crystalline 4-chloro-N-(4-(morpholinomethyl)phenyl)benzamide phosphate salt has the structure of Form 4A.

According to the present invention, crystalline 4-chloro-N-(4-(morpholinomethyl)phenyl)benzamide phosphate salt has the structure of Form 4C.

According to the present invention, a pharmaceutical composition comprises a crystalline phosphate salt according to the present invention and a pharmaceutically acceptable vehicle.

According to the present invention, a method of treating a disease in a patient comprises administering to a patient in need of such treatment a therapeutically effective amount of a crystalline phosphate salt according to the present invention or a pharmaceutical composition according to the present invention, wherein the disease is selected from cancer, an inflammatory disease, an autoimmune disease, a respiratory disease, and an age-related disease.

According to the present invention, a method of treating a disease in a patient comprises administering to a patient in need of such treatment a therapeutically effective amount of a crystalline phosphate salt according to the present invention or a pharmaceutical composition according to the present invention, wherein the disease is selected from acute lung injury, acute respiratory distress syndrome (ARDS), and chronic obstructive pulmonary disease (COPD).

According to the present invention, a method of treating a disease in a patient comprises administering to a patient in need of such treatment a therapeutically effective amount of a crystalline phosphate salt according to the present invention or a pharmaceutical composition according to the present invention, wherein the disease is treated by modulating the p38α MAPK receptor.

According to the present invention, a method of modulating the p38α MAPK receptor in a patient comprises administering to a patient a pharmacologically effective amount of a crystalline phosphate salt according to the present invention or a pharmaceutical composition according to the present invention.

According to the present invention, a kit comprises a crystalline phosphate salt according to the present invention or a pharmaceutical composition according to the present invention.

### BRIEF DESCRIPTION OF THE DRAWINGS

Those skilled in the art will understand that the drawings described herein are for illustration purposes only. The drawings are not intended to limit the scope of the present disclosure.

FIG. 1 shows an optical micrograph of crystalline phosphate salt Form 4A.

FIG. 2 shows an X-ray powder diffraction (XRPD) pattern of crystalline phosphate salt Form 4A.

FIG. 3 shows a single crystal XRPD pattern of crystalline phosphate salt Form 4A.

FIGS. 4-6 show differential scanning calorimeter (DSC) profiles and thermogravimetric analysis (TGA) curves for different batches of crystalline phosphate salt Form 4A.

FIG. 7 shows a DSC profile for crystalline phosphate salt Form 4A.

FIGS. 8A and 8B show a dynamic vapor sorption (DVS) isotherm for crystalline phosphate salt Form 4A and the corresponding DVS kinetic profile, respectively.

FIG. 9 shows an X-ray powder diffraction (XRPD) pattern of crystalline phosphate salt Form 4C.

FIG. 10 shows a differential scanning calorimeter (DSC) profile and a thermogravimetric analysis (TGA) curve for crystalline phosphate salt Form 4C.

FIG. 11 shows a unit cell image of crystalline phosphate salt Form 4A at 100K.

FIG. 12 shows a unit cell image of crystalline phosphate salt Form 4A at room temperature (23° C.)

FIG. 13 shows the crystalline phosphate salt Form 4A in the asymmetric unit.

### DETAILED DESCRIPTION

For purposes of the following detailed description, it is to be understood that embodiments provided by the present disclosure may assume various alternative variations and step sequences, except where expressly specified to the contrary. Moreover, other than in any operating examples, or where otherwise indicated, all numbers expressing, for example, quantities of ingredients used in the specification and claims are to be understood as being modified in all instances by the term “about.” Accordingly, unless indicated to the contrary, the numerical parameters set forth in the following specification and attached claims are approximations that may vary depending upon the desired properties to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard variation found in their respective testing measurements.

Also, it should be understood that any numerical range recited herein is intended to include all sub-ranges. For example, a range of “1 to 10” is intended to include all sub-ranges between (and including) the recited minimum value of 1 and the recited maximum value of 10, that is, having a minimum value equal to or greater than 1 and a maximum value of equal to or less than 10. A sub-range can include, for example, a range from 1 to 5, from 2 to 7, or from 7 to 10.

“A compound provided by the present disclosure” refers to a crystalline 4-chloro-N-(4-(morpholinomethyl)phenyl)benzamide phosphate salt.

A crystalline 4-chloro-N-(4-(morpholinomethyl)phenyl)benzamide phosphate salt provided by the present disclosure refers to the crystalline 4-chloro-N-(4-(morpholinomethyl)phenyl)benzamide phosphate salt having Form 4A, the crystalline 4-chloro-N-(4-(morpholinomethyl)phenyl)benzamide phosphate salt having Form 4C, or a combination thereof.

“Compounds” including derivatives and moieties disclosed herein include any specific compounds within the disclosed formula. Compounds may be identified either by chemical structure and/or by chemical name. Compounds are named using the ChemDraw Professional 22.2.0.3300 (PerkinElmer Informatics, Inc.) nomenclature program. When the chemical structure and chemical name conflict, the chemical structure is determinative of the identity of the compound. The compounds described herein may comprise one or more stereogenic centers and/or double bonds and therefore may exist as stereoisomers such as double-bond isomers (i.e., geometric isomers), enantiomers, diastereomers, or atropisomers. Accordingly, any chemical structures within the scope of the specification depicted, in whole or in part, with a relative configuration encompass all possible enantiomers and stereoisomers of the illustrated compounds including the stereoisomerically pure form (e.g., geometrically pure, enantiomerically pure, or diastereomerically pure) and enantiomeric and stereoisomeric mixtures. Enantiomeric and stereoisomeric mixtures may be resolved into their component enantiomers or stereoisomers using separation techniques or chiral synthesis techniques well known to the skilled in the art.

Compounds including derivatives and moieties disclosed herein include optical isomers of compounds and moieties, racemates thereof, and other mixtures thereof. In such embodiments, the single enantiomers or diastereomers may be obtained by asymmetric synthesis or by resolution of the racemates. Resolution of the racemates may be accomplished, for example, by conventional methods such as crystallization in the presence of a resolving agent, or chromatography, using, for example a chiral high-pressure liquid chromatography (HPLC) column with chiral stationary phases. In addition, compounds include (Z)- and (E)-forms (or cis- and trans-forms) of compounds with double bonds either as single geometric isomers or mixtures thereof.

Compounds including derivatives and moieties may also exist in several tautomeric forms including the enol form, the keto form, and mixtures thereof. Accordingly, the chemical structures depicted herein encompass all possible tautomeric forms of the illustrated compounds. Compounds may exist in unsolvated forms as well as solvated forms, including hydrated forms. Certain compounds may exist in multiple crystalline, co-crystalline, or amorphous forms. Compounds include pharmaceutically acceptable salts thereof, or pharmaceutically acceptable solvates of the free acid form of any of the foregoing, as well as crystalline forms of any of the foregoing.

“Immediate release” refers to a pharmaceutical composition that releases substantially all of a pharmaceutically active ingredient into the gastrointestinal tract of a patient within less than 1 hour following oral administration, such as within less than 50 minutes, within less than 40 minutes, within less than 30 minutes, within less than 20 minutes, or within less than 10 minutes following oral administration. For example, an immediate release dosage form can release greater than 90%, greater than 95%, or greater than 98% of the pharmaceutically active ingredient in the pharmaceutical composition into the gastrointestinal tract within less than 1 hour such as within less than 50 minutes, less than 40 minutes, less than 30 minutes, less than 20 minutes, or less than 10 minutes, following oral administration. Immediate release pharmaceutical compositions can be appropriate to administer pharmaceutically active ingredients that are absorbed into the systemic circulation from the upper portion of the gastrointestinal tract.

“Modified release” pharmaceutical compositions include controlled release formulations, delayed release formulations, extended-release formulations, sustained release formulations, timed release formulations, pulsatile release formulations, and pH-dependent release formulations. These formulations are intended to release a pharmaceutically active ingredient from the pharmaceutical composition at a desired rate and/or at a desired time following oral administration by a patient and/or at a certain location or locations within the gastrointestinal tract and/or at a certain pH within the gastrointestinal tract. The USP defines a modified release system as one in which the time course or location of drug release or both, are chosen to accomplish objectives of therapeutic effectiveness or convenience not fulfilled by immediate release dosage forms. A modified release oral dosage form can include extended release and delayed-release components. A delayed release dosage form is one that releases a drug all at once at a time other than promptly after administration. A modified release formulation can include delayed-release using enteric coatings, site-specific or timed release such as for colonic delivery, extended-release including, for example, formulations capable of providing zero-order, first-order, or biphasic release profiles, and programmed release such as pulsatile and delayed extended release.

“Sustained release” pharmaceutical compositions and coatings provide for a dissolution rate over an extended period of time following oral administration. Granulations comprising granules having a sustained release coating can be referred to as sustained release granulations. A pharmaceutical composition comprising a sustained release granulation can be referred to as a sustained release pharmaceutical composition.

“pH-release” pharmaceutical compositions and coatings provide for an increased dissolution rate at an intended pH.

“Pulsatile release” pharmaceutical compositions and coatings exhibit an increased dissolution rate at intervals, where the release intervals can be determined by time, exposure to internal stimuli, or exposure to external stimuli. Examples of pulsatile-release systems include capsular systems, osmotic systems, systems having erodible membranes, and systems having a rupturable coating. Examples of stimuli include temperature, chemicals, electrical stimuli, and magnetic stimuli.

“Timed-release” pharmaceutical compositions and coatings have a dissolution rate that is a function of time. A time-release pharmaceutical composition or coating includes, for example, delayed release, sustained release, and extended release pharmaceutical compositions and coatings.

“Delayed release” pharmaceutical compositions and coatings provide for an increased dissolution rate at an intended time after administration.

“Modulate” and “modulation” refer to a change in biological activity for a biological molecule such as, for example, a protein, gene, peptide, or antibody, where such change may relate to an increase in biological activity such as, for example, increased activity, agonism, activation, expression, upregulation, and/or increased expression, or decrease in biological activity such as, for example, decreased activity, antagonism, suppression, deactivation, downregulation, and/or decreased expression, for the biological molecule. For example, compound (2) can modulate the p38α MAPK protein such as inhibit p38α MAPK protein. Compound (2) can selectively modulate such as selectively inhibit p38α MAPK protein as compared to other MAPK or p38 MAPK proteins.

“Patient” refers to a mammal, for example, a human.

“Pharmaceutically acceptable” refers to approved or approvable by a regulatory agency of the Federal or a state government or listed in the U.S. Pharmacopoeia or other generally recognized pharmacopoeia for use in animals, and more particularly in humans.

“Pharmaceutically acceptable vehicle” refers to a pharmaceutically acceptable diluent, a pharmaceutically acceptable adjuvant, a pharmaceutically acceptable excipient, a pharmaceutically acceptable carrier, or a combination of any of the foregoing with which a compound provided by the present disclosure may be administered to a patient and which does not destroy the pharmacological activity thereof and which is non-toxic when administered in doses sufficient to provide a therapeutically effective amount of the compound.

A “pharmaceutically acceptable salt” such as a phosphate salt includes hydrates and other solvates, as well as salts in crystalline or non-crystalline form. Where a particular pharmaceutically acceptable salt is disclosed, it is understood that the particular salt (e.g., a hydrochloride salt) is an example of a salt, and that other salts may be formed using techniques known to one of skill in the art. Additionally, one of skill in the art would be able to convert the pharmaceutically acceptable salt to the corresponding compound, free base and/or free acid, using techniques generally known in the art.

“Pharmaceutical composition” refers to a composition comprising compound provided by the present disclosure and at least one pharmaceutically acceptable vehicle suitable for administration to a patient for treating a disease or disorder. A pharmaceutically acceptable vehicle refers to a diluent, excipient or carrier with which a compound is administered to a patient. Pharmaceutically acceptable vehicles are known in the art. “A pharmaceutical composition can be administered to a patient.

“Disease” refers to a disease, disorder, condition, or symptom of any of the foregoing.

“Preventing” or “prevention” refers to a reduction in risk of acquiring a disease or disorder (i.e., causing at least one of the clinical symptoms of the disease not to develop in a patient that may be exposed to or predisposed to the disease but does not yet experience or display symptoms of the disease). In some embodiments, “preventing” or “prevention” refers to reducing symptoms of the disease by administering a compound provided by the present disclosure in a preventative fashion. The application of a therapeutic agent for preventing or prevention of a disease of disorder is known as ‘prophylaxis.’ Compounds provided by the present disclosure can provide superior prophylaxis because of lower long-term side effects over long time periods.

“Curing” a disease refers to eliminating a disease or disorder or eliminating a symptom of a disease or disorder.

“Treating” or “treatment” of a disease or disorder refers to inhibiting the disease or disorder or one or more clinical symptoms of the disease or disorder, arresting the development of the disease or disorder or one or more clinical symptoms of the disease or disorder, relieving the disease or disorder or one or more clinical symptoms of the disease or disorder, causing the regression of the disease or disorder or one or more clinical symptoms of the disease or disorder, reducing the severity of one or more clinical symptom of the disease or disorder, delaying the onset of one or more clinical symptoms of the disease or disorder, mitigating one or more clinical symptoms of the disease or disorder and/or stabilizing the disease or disorder or one or more clinical symptoms of the disease or disorder. “Treating” or “treatment” of a disease or disorder includes producing a clinically beneficial effect without curing the underlying disease or disorder.

“Therapeutically effective amount” refers to the amount of a compound such as pharmaceutically active ingredient that, when administered to a patient for treating a disease, or at least one of the clinical symptoms of a disease, is sufficient to affect such treatment of the disease or symptom thereof. A “therapeutically effective amount” may vary depending, for example, on the compound, the disease and/or symptoms of the disease, the severity of the disease and/or symptoms of the disease or disorder, the age, weight, and/or health of the patient to be treated, and the judgment of the prescribing physician. A therapeutically effective amount in any given instance may be ascertained by those skilled in the art or capable of determination by routine experimentation.

“Therapeutically effective dose” refers to a dose that provides effective treatment of a disease or disorder in a patient. A therapeutically effective dose may vary from compound to compound, and from patient to patient, and may depend upon factors such as the condition of the patient and the route of delivery. A therapeutically effective dose may be determined in accordance with routine pharmacological procedures known to those skilled in the art.

4-Chloro-N-(4-(morpholinomethyl)phenyl)benzamide has the chemical structure of Formula (1):

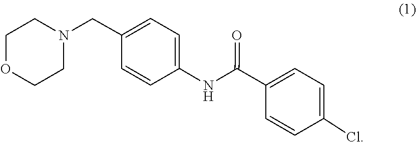 [SMILES: O=C(Nc1ccc(CN2CCOCC2)cc1)c1ccc(Cl)cc1]

4-Chloro-N-(4-(morpholinomethyl)phenyl)benzamide is also referred to as the free base.

Crystalline 4-chloro-N-(4-(morpholinomethyl)phenyl)benzamide hydrochloride is a stable form of the free base and is disclosed in U.S. Pat. No. 12,116,353.

Compounds provided by the present disclosure are crystalline phosphoric acid salts of 4-chloro-N-(4-(morpholinomethyl)phenyl)benzamide.

4-Chloro-N-(4-(morpholinomethyl)phenyl)benzamide phosphate and crystalline forms thereof can be characterized by methods known in the art such as optical microscopy, thermal analysis including differential scanning calorimetry (DSC), thermal gravimetric analysis (TGA), 1H nuclear magnetic resonance (1H NMR), X-ray powder diffraction (XRPD), solid state stability and hygroscopicity by dynamic vapor sorption (DVS) followed by post-DVS PXRD analysis, solubility, and purity.

4-Chloro-N-(4-(morpholinomethyl)phenyl)benzamide phosphate salt provided by the present disclosure can have Form 4A or Form 4C. A phosphate salt can have the structure of Formula (2):

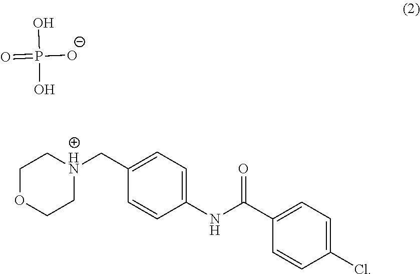

Crystalline 4-chloro-N-(4-(morpholinomethyl)phenyl)benzamide phosphate salt can have Form 4A.

Crystalline 4-chloro-N-(4-(morpholinomethyl)phenyl)benzamide phosphate Form 4A is described in Example 2.

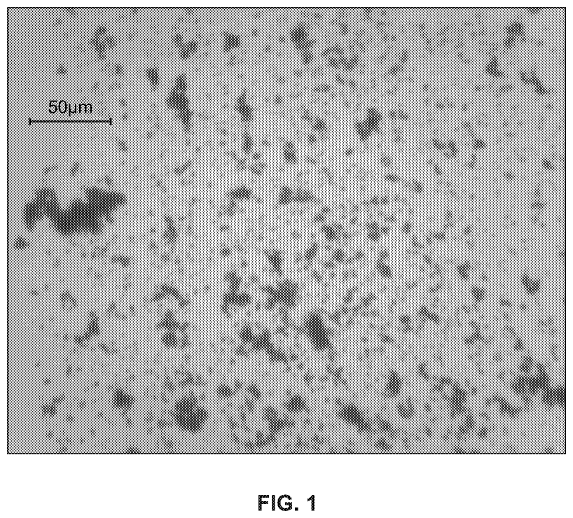

An optical micrograph of crystalline 4-chloro-N-(4-(morpholinomethyl)phenyl)benzamide phosphate Form 4A is shown in FIG. 1 where the crystal morphology is characterized by granular-like, irregular agglomerates, and fine particles.

Crystalline phosphate salt Form 4A can be characterized by an XRPD pattern comprising characteristic diffraction peaks at least at 7.9°±0.2°, 16.9°±0.2°, 17.9°±0.2°, 18.6°±0.2°, 21.7°±0.2°, 23.8°±0.2°, 24.1°±0.2°, 26.2°±0.2°, and 27.1°±0.2°, expressed as θ/2θ angles and determined using Cu-Kα radiation.

Crystalline phosphate salt Form 4A can be characterized by an XRPD pattern comprising characteristic diffraction peaks at least at 7.9°±0.1°, 16.9°±0.1°, 17.9°±0.1°, 18.6°±0.1°, 21.7°±0.1°, 23.8°±0.1°, 24.1°±0.1°, 26.2°±0.1°, and 27.1°±0.1°, expressed as θ/2θ angles and determined using Cu-Kα radiation.

Crystalline phosphate salt Form 4A can be characterized by an XRPD pattern comprising characteristic diffraction peaks at least at 7.9°±0.2°, 8.5°±0.2°, 13.7°±0.2°, 16.1°±0.2°, 16.8°±0.2°, 16.9°±0.2°, 17.9°±0.2°, 18.6°±0.2°, 21.2°±0.2°, 21.7°±0.2°, 23.8°±0.2°, 24.1°±0.2°, 24.9°±0.2°, 25.9°±0.2°, 26.2°±0.2°, 27.1°±0.2°, and 30.3°±0.2°, expressed as θ/2θ angles and determined using Cu-Kα radiation.

Crystalline phosphate salt Form 4A can be characterized by an XRPD pattern comprising characteristic diffraction peaks at least at 7.9°±0.1°, 8.5°±0.1°, 13.7°±0.1°, 16.1°±0.1°, 16.8°±0.1°, 16.9°±0.1°, 17.9°±0.1°, 18.6°±0.1°, 21.2°±0.1°, 21.7°±0.1°, 23.8°±0.1°, 24.1°±0.1°, 24.9°±0.1°, 25.9°±0.1°, 26.2°±0.1°, 27.1°±0.1°, and 30.3°±0.1°, expressed as θ/2θ angles and determined using Cu-Kα radiation.

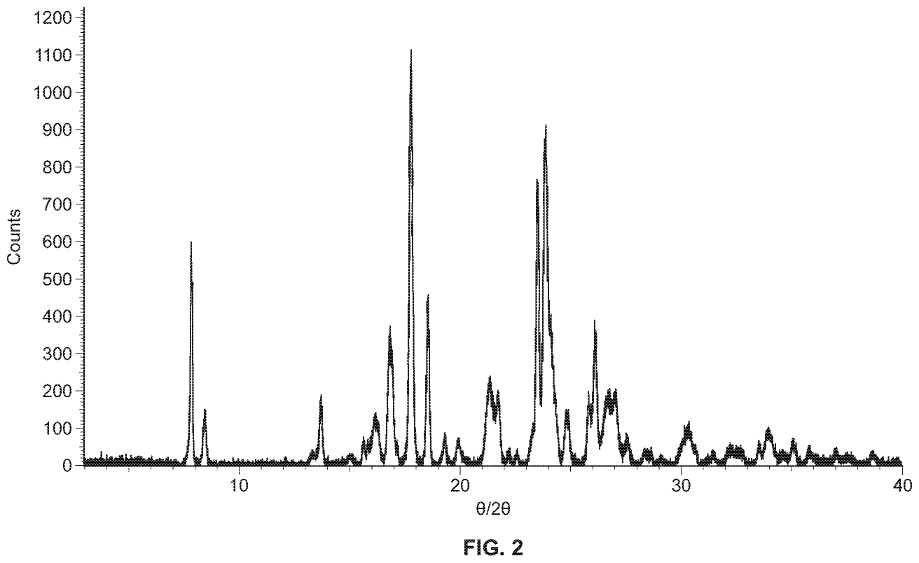

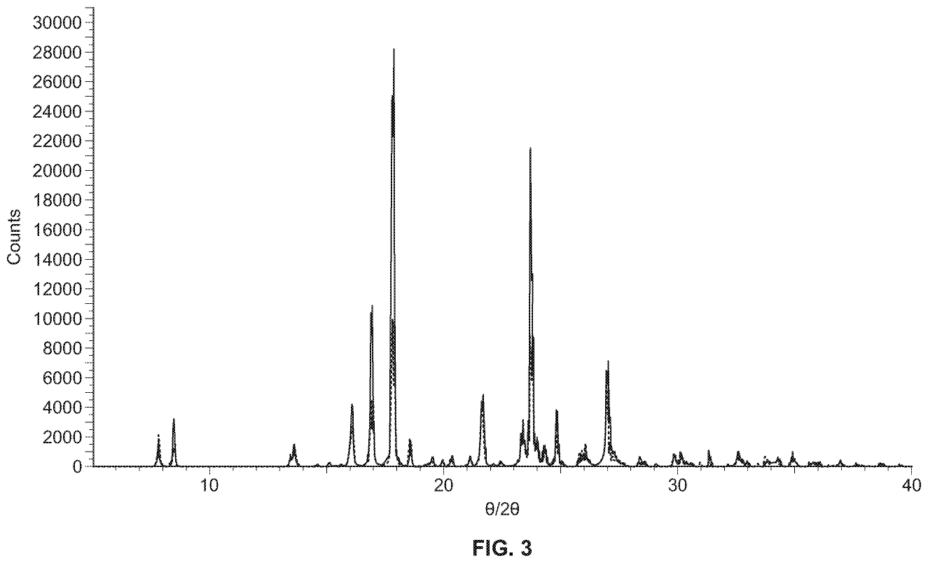

Crystalline phosphate salt Form 4A can be characterized by an XRPD pattern substantially as shown in FIG. 2 and in FIG. 3.

Crystalline 4-chloro-N-(4-(morpholinomethyl)phenyl)benzamide phosphate Form 4A can have a differential scanning calorimetry curve characterized by a sharp endotherm with an onset temperature from 219° C. to 230° C., such as from 221° C. to 228° C., or from 223° C. to 225° C.

Crystalline 4-chloro-N-(4-(morpholinomethyl)phenyl)benzamide phosphate Form 4A can have a differential scanning calorimetry curve characterized by an exothermic peak from 229° C. to 232° C.

Crystalline 4-chloro-N-(4-(morpholinomethyl)phenyl)benzamide phosphate Form 4A can have a differential scanning calorimetry curve characterized by an exothermic enthalpy from 137 J/g to 147 J/g, such as from 139 J/g to 145 J/g, or from 141 J/g to 143 J/g.

Crystalline 4-chloro-N-(4-(morpholinomethyl)phenyl)benzamide phosphate Form 4A can have a differential scanning calorimetry curve characterized by a broad endotherm with an onset temperature from 165° C. to 174° C., such as from 167° C. to 172° C.

Crystalline 4-chloro-N-(4-(morpholinomethyl)phenyl)benzamide phosphate Form 4A can have a differential scanning calorimetry curve characterized by an endothermic peak from 170° C. to 180° C., such as from 172° C. to 178° C.

Crystalline 4-chloro-N-(4-(morpholinomethyl)phenyl)benzamide phosphate Form 4A can have a differential scanning calorimetry curve characterized by an endothermic enthalpy from 1 J/g to 3 J/g, such as from 1.5 J/g to 2.5 J/g.

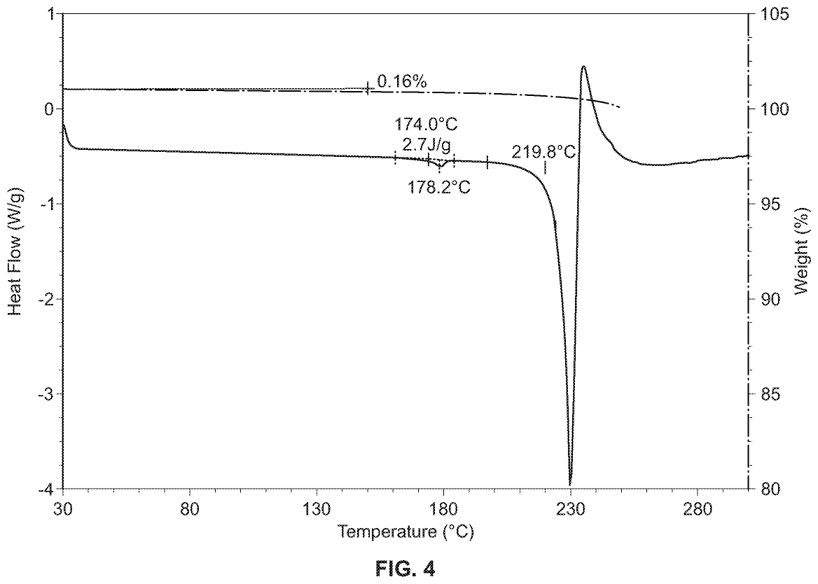

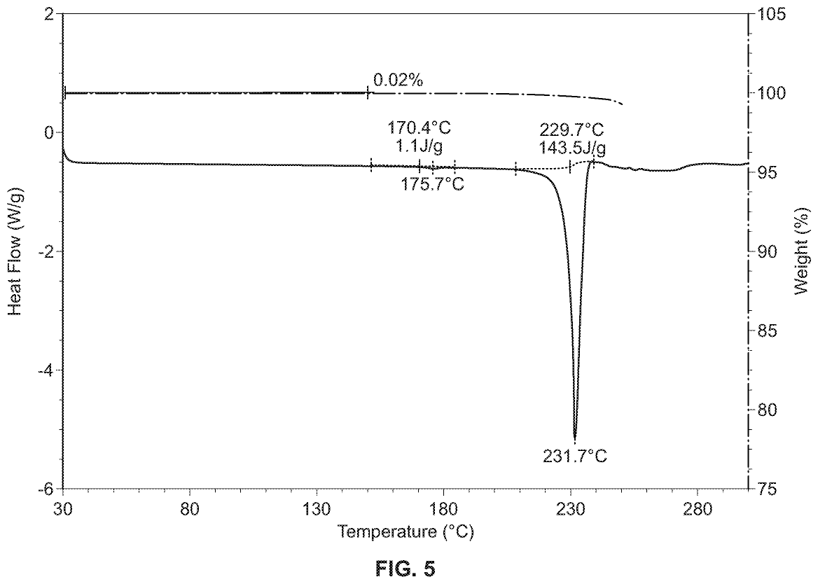

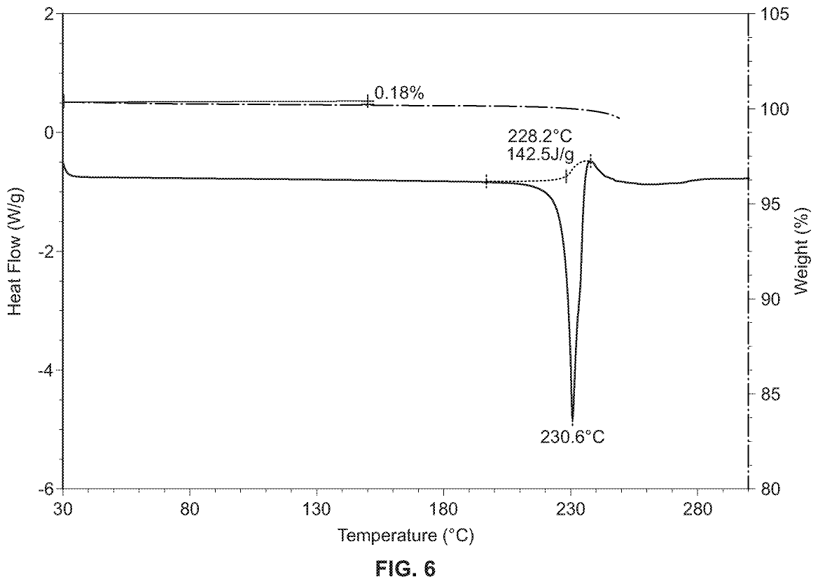

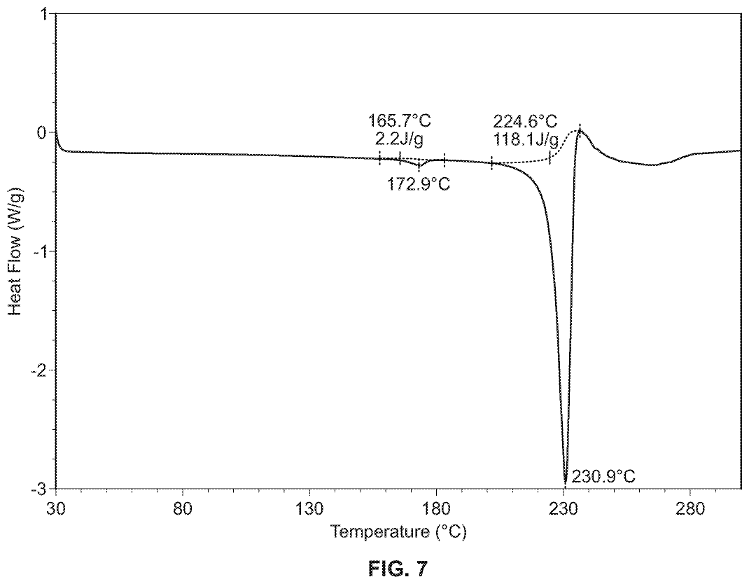

Crystalline 4-chloro-N-(4-(morpholinomethyl)phenyl)benzamide phosphate Form 4A can have a DSC profile substantially as shown in FIGS. 4-7.

Crystalline 4-chloro-N-(4-(morpholinomethyl)phenyl)benzamide phosphate Form 4A can have a TGA profile characterized by a weight loss of from 0% to 0.25%, such as from 0.11% to 0.21%, or from about 0.15% to 0.20% within a temperature range from 30° C. to 150° C., and with decomposition at higher temperatures.

Crystalline 4-chloro-N-(4-(morpholinomethyl)phenyl)benzamide phosphate Form 4A can have a thermogravimetric analysis curve substantially as shown in FIGS. 4-6.

Crystalline 4-chloro-N-(4-(morpholinomethyl)phenyl)benzamide phosphate Form 4A can exhibit a weight gain of less than 2.5% from 0% RH to 95% RH as determined by dynamic vapor sorption.

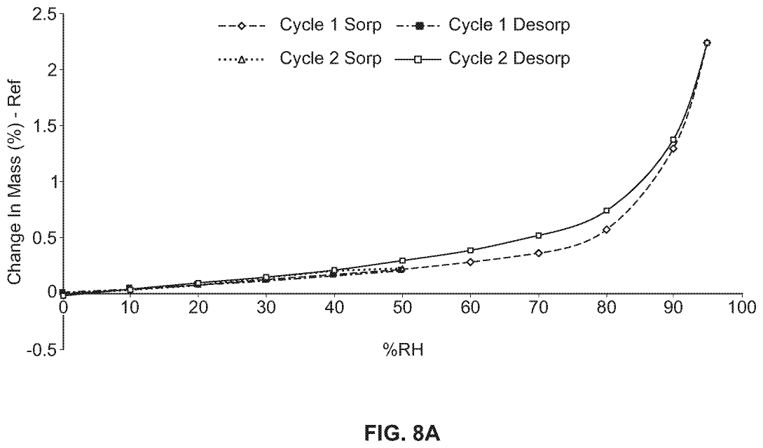

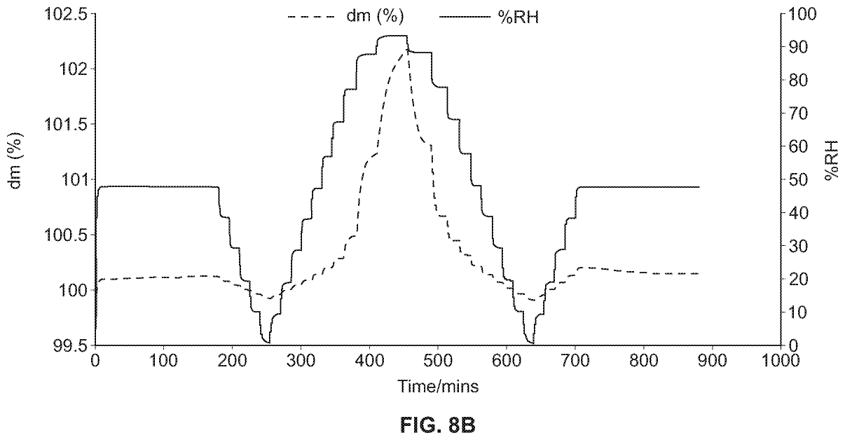

Crystalline 4-chloro-N-(4-(morpholinomethyl)phenyl)benzamide phosphate Form 4A can have a dynamic vapor sorption isotherm plot as substantially shown in FIG. 8A.

Crystalline 4-chloro-N-(4-(morpholinomethyl)phenyl)benzamide phosphate Form 4A can have a solubility in a 1:1 ACN:water mixture from 50 mg/mL to 60 mg/mL at 40° C.

Crystalline 4-chloro-N-(4-(morpholinomethyl)phenyl)benzamide phosphate Form 4A can have a solubility in a 1:1 THF:water mixture from 75 mg/mL to 85 mg/mL at 40° C.

Crystalline 4-chloro-N-(4-(morpholinomethyl)phenyl)benzamide phosphate Form 4A can be stable following exposure to 40° C./75% RH for at least one week.

Crystalline 4-chloro-N-(4-(morpholinomethyl)phenyl)benzamide phosphate Form 4A can have a purity, for example, greater than 90 wt %, greater than 92 wt %, greater than 94 wt %, greater than 96 wt %, greater than 98 wt %, greater than 99 wt %, greater than 99.5 wt %, or greater than 99.9 wt %, where wt % is based on the total weight of the crystalline 4-chloro-N-(4-(morpholinomethyl)phenyl)benzamide phosphate Form 4A.

Crystalline 4-chloro-N-(4-(morpholinomethyl)phenyl)benzamide phosphate salt can have Form 4C.

Crystalline phosphate salt Form 4C can be characterized by an XRPD pattern comprising characteristic diffraction peaks at least at 13.7°±0.2°, 17.3°±0.2°, 19.8°±0.2°, 20.5°±0.2°, 22.6°±0.2°, and 25.1°±0.2°, expressed as θ/2θ angles and determined using Cu-Kα radiation.

Crystalline phosphate salt Form 4C can be characterized by an XRPD pattern comprising characteristic diffraction peaks at least at 13.7°±0.1°, 17.3°±0.1°, 19.8°±0.1°, 20.5°±0.1°, 22.6°±0.1°, and 25.1°±0.1°, expressed as θ/2θ angles and determined using Cu-Kα radiation.

Crystalline phosphate salt Form 4C can be characterized by an XRPD pattern comprising characteristic diffraction peaks at least at 13.5°±0.2°, 13.7°±0.2°, 15.9°±0.2°, 17.3°±0.2°, 19.5°±0.2°, 19.8°±0.2°, 20.5°±0.2°, 22.6°±0.2°, 24.0°±0.2°, 24.4°±0.2°, and 25.1°±0.2°, expressed as θ/2θ angles and determined using Cu-Kα radiation.

Crystalline phosphate salt Form 4C can be characterized by an XRPD pattern comprising characteristic diffraction peaks at least at 13.5°±0.1°, 13.7°±0.1°, 15.9°±0.1°, 17.3°±0.1°, 19.5°±0.1°, 19.8°±0.1°, 20.5°±0.1°, 22.6°±0.1°, 24.0°±0.1°, 24.4°±0.1°, and 25.1°±0.1°, expressed as θ/2θ angles and determined using Cu-Kα radiation.

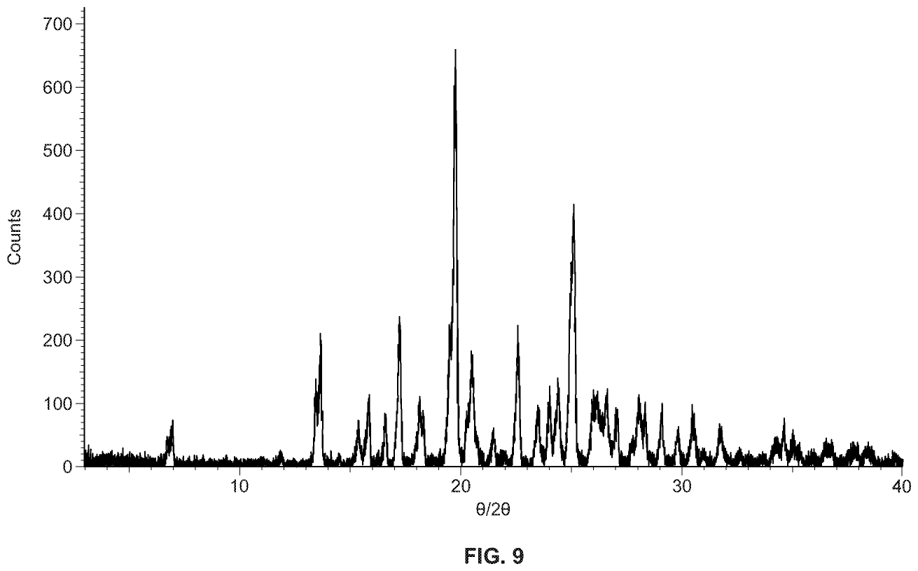

Crystalline 4-chloro-N-(4-(morpholinomethyl)phenyl)benzamide phosphate Form 4C can have an XRPD pattern substantially as shown in FIG. 9.

Crystalline 4-chloro-N-(4-(morpholinomethyl)phenyl)benzamide phosphate Form 4C can have a DSC profile characterized by a small endotherm with an onset temperature at 34.1° C.±5° C. or 34.1° C.±2.0° C., followed by a broad endotherm with an onset temperature at 153.4° C.±5.0° C. or 153.4° C.±2.0° C. The broad endotherm can have a peak temperature at 172.0° C.±5.0° C. or 170.0° C.±2.0° C.

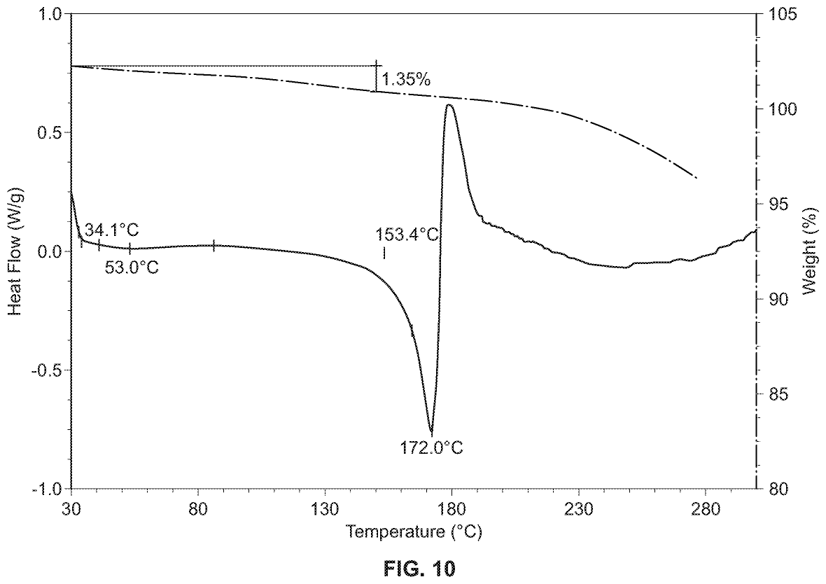

Crystalline 4-chloro-N-(4-(morpholinomethyl)phenyl)benzamide phosphate Form 4C can have a DSC profile substantially as shown in FIG. 10.

Crystalline 4-chloro-N-(4-(morpholinomethyl)phenyl)benzamide phosphate Form 4C can have a TGA profile characterized by a weight loss of from 0.5% to 2.0%, such as from 1.0% to 1.5%, or about 1.35% within a temperature range from 30° C. to 180° C., and with decomposition at higher temperatures.

Crystalline 4-chloro-N-(4-(morpholinomethyl)phenyl)benzamide phosphate Form 4C can have a TGA profile substantially as shown in FIG. 10.

Crystalline 4-chloro-N-(4-(morpholinomethyl)phenyl)benzamide phosphate Form 4C can have a purity, for example, greater than 90 wt %, greater than 92 wt %, greater than 94 wt %, greater than 96 wt %, greater than 98 wt %, greater than 99 wt %, greater than 99.5 wt %, or greater than 99.9 wt %, where wt % is based on the total weight of the crystalline 4-chloro-N-(4-(morpholinomethyl)phenyl)benzamide phosphate Form 4C.

A pharmaceutical composition provided by the present disclosure can comprise a crystalline 4-chloro-N-(4-(morpholinomethyl)phenyl)benzamide phosphate salt provided by the present disclosure.

A pharmaceutical composition can comprise a therapeutically effective amount of a crystalline phosphate salt provided by the present disclosure for treating a disease in a patient.

A pharmaceutical composition can comprise one or more pharmaceutically acceptable carriers, excipients diluents, or combinations of any of the foregoing.

A crystalline phosphate salt provided by the present disclosure can be incorporated into a pharmaceutical composition to be administered to a patient by any appropriate route of administration including intradermal, intramuscular, intraperitoneal, intravenous, subcutaneous, intranasal, epidural, oral, peroral, sublingual, intracerebral, intravaginal, transdermal, rectal, inhalation, or topical. A pharmaceutical composition provided by the present disclosure can be an injectable formulation. A pharmaceutical composition may be formulated for intravenous administration or for subcutaneous administration. Pharmaceutical compositions provided by the present disclosure can be injectable intravenous formulations. A pharmaceutical composition provided by the present disclosure can be oral formulations. Oral formulations may be oral dosage forms.

Pharmaceutical compositions provided by the present disclosure may comprise a therapeutically effective amount of a crystalline phosphate salt together with a suitable amount of one or more pharmaceutically acceptable vehicles so as to provide a composition for proper administration to a patient. Suitable pharmaceutically acceptable vehicles and methods of preparing pharmaceutical compositions are described in the art.

A pharmaceutical composition provided by the present disclosure can be formulated for oral administration. A pharmaceutical composition formulated for oral administration can comprise any suitable oral dosage form including, for example, tablets, capsules, caplets, sachets, bottles, stick packs, dispersions, and suspensions.

A pharmaceutical composition formulated for oral administration can provide for a modified release profile in the gastrointestinal tract, such as a controlled release profile, a sustained release profile, a pH-release profile, a pulsatile release profile, a timed-release profile, or a delayed release profile. A pharmaceutical composition formulated for oral administration can be configured to release a crystalline compound over an intended period of time following ingestion and/or in an intended region of the gastrointestinal tract.

A pharmaceutical composition formulated for oral administration can provide for an immediate release profile.

Assessing single patient response to therapy and qualifying a patient for optimal therapy are among the greatest challenges of modern healthcare and relate to trends in personalized medicine. 4-Chloro-N-(4-(morpholinomethyl)phenyl)benzamide can have target selectivity, for example, for certain cancers and immune cells. A phosphate salt provided by the present disclosure radiolabeled for positron emission tomography (PET) or Single Photon Emission Computed Tomography (SPECT) can be used to predict the targeting of the treatment based on a single-study, case-by-case patient analysis thus excluding patients that are expected not to benefit from treatment. PET/SPECT scans using a phosphate salt provided by the present disclosure, once correlated to the concentration can provide a three-dimensional distribution map, which can then be used for macroscopic dose calculations.

Accordingly, it is within the capability of those of skill in the art to assay and use a crystalline phosphate salt provided by the present disclosure and/or a pharmaceutical composition thereof for therapy.

A crystalline phosphate salt provided by the present disclosure and/or pharmaceutical composition thereof can generally be used in an amount effective to achieve the intended purpose. For use to treat a disease such as cancer, an autoimmune disease a respiratory disease, an age-related disease, or an inflammatory disease, a phosphate salt provided by the present disclosure can be administered or applied in a therapeutically effective amount.

The amount of a crystalline phosphate salt provided by the present disclosure that will be effective in the treatment of a particular disorder or condition disclosed herein can depend in part on the nature of the disorder or condition, and can be determined by standard clinical techniques known in the art. In addition, in vitro or in vivo assays may optionally be employed to help identify optimal dosage ranges. The suitable amount of a crystalline phosphate salt provided by the present disclosure can depend on, among other factors, the patient being treated, the weight of the patient, the severity of the affliction, the manner of administration and the judgment of the prescribing physician.

A phosphate salt provided by the present disclosure can be assayed in vitro and in vivo, for the desired therapeutic activity, prior to use in humans. For example, in vitro assays may be used to determine whether administration of a specific compound or a combination of compounds is preferred. The compounds can also be demonstrated to be effective and safe using animal model systems.

A therapeutically effective dose of a crystalline phosphate salt provided by the present disclosure and/or pharmaceutical composition thereof will provide therapeutic benefit without causing substantial toxicity. Toxicity of a crystalline phosphate salt provided by the present disclosure and/or pharmaceutical composition thereof may be determined using standard pharmaceutical procedures and may be readily ascertained by the skilled artisan. The dose ratio between toxic and therapeutic effect can be referred to as the therapeutic index. A crystalline phosphate salt provided by the present disclosure and/or pharmaceutical composition thereof exhibits a particularly high therapeutic index in treating disease and disorders. A dose of a crystalline phosphate salt provided by the present disclosure and/or pharmaceutical composition thereof can be within a range of circulating concentrations that include an effective dose with minimal toxicity.

A crystalline phosphate salt provided by the present disclosure, or a pharmaceutical composition thereof may be included in a kit that may be used to administer the compound to a patient for therapeutic purposes. A kit can include a pharmaceutical composition comprising a crystalline phosphate salt provided by the present disclosure suitable for administration to a patient and instructions for administering the pharmaceutical composition to the patient. A kit can be suitable for treating cancer, for treating an autoimmune disease, for treating an age-related disease, for treating a respiratory disease, or for treating an inflammatory disease. A kit can comprise a crystalline phosphate salt provided by the present disclosure, a pharmaceutically acceptable vehicle for administering the phosphate salt, and instructions for administering the formulation comprising the crystalline phosphate salt to a patient.

The pharmaceutical compositions can be included, for example, in a container, blister pack, packet, sachet, or dispenser together with instructions for administration.

Instructions supplied with a kit may be printed and/or supplied, for example, as an electronic-readable medium, a video cassette, an audiotape, a flash memory device, or may be published on an internet web site or distributed to a patient and/or health care provider as an electronic communication.

A crystalline phosphate salt provided by the present disclosure and a pharmaceutical composition comprising a phosphate salt provided by the present disclosure can be used to treat a disease in which the etiology of the disease is associated with p38α MAPK protein activity.

4-Chloro-N-(4-(morpholinomethyl)phenyl)benzamide is a selective inhibitor of p38α MAPK. A selective p38α MAPK inhibitor has a higher binding affinity to the target pocket of p38α MAPK than to the catalytic binding site of p38α MAPK. A p38α MAPK inhibitor can bind to p38α MAPK near the substrate binding groove of p38α MAPK, which extends between two acidic regions, the CD and ED domains. The binding pocket can be defined at least by residues R49, H107, L108, and K165 of p38α MAPK. The binding pocket can be defined at least by residues R49, H107, L108, M109, G110, A157, V158, El 63, L164, and K165 of p38α MAPK.

Selective binding of 4-chloro-N-(4-(morpholinomethyl)phenyl)benzamide p38α MAPK can be confirmed using complementary technologies. For example, a selective p38α MAPK inhibitor can show a concentration-dependent increase in melting temperature of p38α but not p38P as determined using DSF, which detects ligand-induced protein stabilization. STD-NMR, which measures low affinity protein/ligand binding via non-scalar magnetization transfer from protein to ligand protons, can be used to confirm specific compound binding to p38α and localized the interaction to its aromatic rings. A p38α MAPK inhibitor can cause a concentration-dependent increase in melting temperature of p38α MAPK. The difference in melting temperature Tₘ can be measured at a p38α MAPK inhibitor concentration of between 1 nM and 1,000 μM such as at a concentration of 100 μM. For example, the difference in the melting temperature can be from 0.1° C. and about 2° C.

4-Chloro-N-(4-(morpholinomethyl)phenyl)benzamide can interact with a pocket near the ED substrate docking site of p38 MAPK.

4-Chloro-N-(4-(morpholinomethyl)phenyl)benzamide can bind to p38α MAPK near the substrate binding groove of p38α MAPK, which extends between the CD and ED domains.

4-Chloro-N-(4-(morpholinomethyl)phenyl)benzamide can inhibit MK2 phosphorylation through interaction with p38α MAPK.

4-Chloro-N-(4-(morpholinomethyl)phenyl)benzamide can competitively bind to p38α MAPK with 4-chloro-N-(4-((1,1-dioxidothiomorpholino)methyl)phenyl)benzamide.

4-Chloro-N-(4-(morpholinomethyl)phenyl)benzamide can have a higher binding affinity to the p38α MAPK subunit than to the p38β MAPK subunit.

Phosphorylation of MK2 requires binding to the ED site adjacent to the target pocket in p38α MAPK. The target pocket can be defined by amino acids R49, H107, L108, and K165 in p38α MAPK. The target pocket can be defined by amino acids selected from R49, H107, L108, M109, G110, A157, V158, E163, L164, and K165 in p38α MAPK. The target pocket can be defined by the amino acids R49, H107, L108, M109, G110, A157, V158, E163, L164, and K165 in p38α MAPK.

4-Chloro-N-(4-(morpholinomethyl)phenyl)benzamide can at least partially inhibit MK2 phosphorylation. For example, Western blotting can be used to measure inhibition of MK2 phosphorylation in anisomycin-stimulated HeLa cells by 4-chloro-N-(4-(morpholinomethyl)phenyl)benzamide.

4-Chloro-N-(4-(morpholinomethyl)phenyl)benzamide can stabilize an endothelial or epithelial barrier function. Endothelial barrier permeability can be measured by separate or combined exposure to TNFa and hyperthermia, followed by measurement of permeably for 10 kDa dextran. For example, endothelial barrier stabilization can be assessed by pretreating with 4-chloro-N-(4-(morpholinomethyl)phenyl)benzamide, preceded and followed by permeability measurements, where stabilization can be expressed as a percent reduction in the before and after pretreatment permeability increase. A permeability increase for 10 kDa dextran can be reduced by between 5% to more than 100% such as, for example, by greater than 5%, greater than 10%, greater than 20%, greater than 40%, greater than 60%, greater than 80%, or greater than 100%.

4-Chloro-N-(4-(morpholinomethyl)phenyl)benzamide can modulate TNFa-induced gene expression in human lung microvascular endothelial cells (HMVECLs) as determined using, for example, RNASeq. For example, HMVECLs can be pretreated for a period of time with a p38α MAPK inhibitor at an appropriate concentration and then stimulated with TNFa for a period of time. 4-Chloro-N-(4-(morpholinomethyl)phenyl)benzamide can inhibit genes such as PRRG4, TSLP, CCLI 7, EXOC3L4, MMP9, IDOI, CXCLIO, CD200, SLCI5A3, VDR, ILIB, GPR88, CD207, TCHH, HAS3, GBPIPI, MUC4, ELOVL7, CXCLII, GBP4, PLAIA, and/or CXCL5.

The effects of a p38α MAPK inhibitor on inflammatory cytokine expression can be determined by pretreating PMA-differentiated THPI cells with a p38α MAPK inhibitor, then stimulating with LPS, and harvesting RNA a period of time later for analysis by PCR-based cytokine array. A p38α MAPK inhibitor can inhibit expression of various genes, such as IL-IA, IL-8, TNFSF8, CXCL5, CCL7, CCLI7, TNFSF9, IL-IB, CXCLI, TNFSFI5, CCL5, CCL4, CCL20, CXCL2, TNF, or BMP6. A p38α MAPK inhibitor can inhibit expression of Smad3, which drives differentiation of Foxp3 T regulatory cells and suppresses interferon-γ. Inflammation reduction can be measured by comparing the fold change mRNA levels vs. unstimulated PMA-differentiated THPI cells at various concentrations of p38α MAPK inhibitor.

Crystalline 4-chloro-N-(4-(morpholinomethyl)phenyl)benzamide phosphate salt provided by the present disclosure or a pharmaceutical composition thereof can be used to treat a disease in a patient.

A crystalline phosphate salt provided by the present disclosure or a pharmaceutical composition thereof can be used to treat a disease in which the etiology of the disease is associated with up-regulation and/or down-regulation of the p38α MAPK protein.

A crystalline phosphate salt provided by the present disclosure or a pharmaceutical composition thereof can be used to treat a disease in which the etiology of the disease is associated with modulation of the p38α MAPK protein.

Methods provided by the present disclosure include treating a disease in a patient comprising administering to a patient in need of such treatment a therapeutically effective amount of a crystalline phosphate salt provided by the present disclosure or a pharmaceutical composition thereof, wherein the disease is treated by inhibiting the p38α MAPK protein.

The p38 mitogen-activated protein kinase (MAPK) family of stress- and cytokine-activated kinases are associated with the pathogenesis of many human diseases, including, for example, cancer, rheumatoid arthritis, cardiovascular disease, multiple sclerosis, inflammatory bowel disease, chronic obstructive pulmonary disease (COPD), asthma, acute respiratory distress syndrome (ARDS), and acute lung injury (ALI). Among the many important biological processes regulated by p38 MAPK, regulation of endothelial and epithelial barrier function, leukocyte trafficking, and cytokine expression are central to the pathogenesis of acute and chronic inflammatory disorders.

A crystalline phosphate salt provided by the present disclosure, or a pharmaceutical composition thereof can be used for treating cancer in a patient. The cancer can be, for example, a solid tumor or a metastasis.

Methods provided by the present disclosure include methods of treating cancer in a patient comprising administering to a patient in need of such treatment a therapeutically effective amount of a crystalline phosphate salt provided by the present disclosure or pharmaceutical composition provided by the present disclosure.

Examples of suitable cancers include acoustic neuroma, adenocarcinoma, angiosarcoma, astrocytoma, basal cell carcinoma, bile duct carcinoma, bladder carcinoma, brain cancer, breast cancer, bronchogenic carcinoma, cervical cancer, chordoma, choriocarcinoma, colon cancer, colorectal cancer, craniopharyngioma, cystadenocarcinoma, embryonal carcinoma, endotheliocarcinoma, ependymoma, epithelial carcinoma, esophageal cancer, Ewing's tumor, fibrosarcoma, gastric cancer, glioblastoma multiforme, glioma, head and neck cancer, hemangioblastoma, hepatoma, kidney cancer, leiomyosarcoma, liposarcoma, lung cancer, lymphangioendotheliosarcoma, lymphangiosarcoma, medullary carcinoma, medulloblastoma, melanoma, meningioma, mesothelioma, myxosarcoma, nasal cancer, neuroblastoma, oligodendroglioma, oral cancer, osteogenic sarcoma, ovarian cancer, pancreatic cancer, papillary adenocarcinoma, papillary carcinoma, pinealoma, prostate cancer, rhabdomyosarcoma, rectal cancer, renal cell carcinoma, retinoblastoma, sarcoma, sebaceous gland carcinoma, seminoma, skin cancer, squamous cell carcinoma, stomach cancer, sweat gland carcinoma, synovioma, testicular cancer, small cell lung carcinoma, throat cancer, uterine cancer, Wilm's tumor, blood cancer, acute erythroleukemic leukemia, acute lymphoblastic B-cell leukemia, acute lymphoblastic T-cell leukemia, acute lymphoblastic leukemia, acute megakaryoblastic leukemia, acute monoblastic leukemia, acute myeloblastic leukemia, acute myelomonocytic leukemia, acute nonlymphocytic leukemia, acute promyelocytic leukemia, acute undifferentiated leukemia, chronic lymphocytic leukemia, chronic myelocytic leukemia, hairy cell leukemia, multiple myeloma, heavy chain disease, and Hodgkin's disease.

Examples of suitable cancers include pancreatic cancer, breast cancer, prostate cancer, lymphoma, skin cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, or retinoblastoma, and the like. In other embodiments, the cancer is acoustic neuroma, adenocarcinoma, angiosarcoma, astrocytoma, basal cell carcinoma, bile duct carcinoma, bladder carcinoma, brain cancer, breast cancer, bronchogenic carcinoma, cervical cancer, chordoma, choriocarcinoma, colon cancer, colorectal cancer, craniopharyngioma, cystadenocarcinoma, embryonal carcinoma, endotheliosarcoma, ependymoma, epithelial carcinoma, esophageal cancer, Ewing's tumor, fibrosarcoma, gastric cancer, glioblastoma multiforme, glioma, head and neck cancer, hemangioblastoma, hepatoma, kidney cancer, leiomyosarcoma, liposarcoma, lung cancer, lymphangioendothelioma, lymphangiosarcoma, medullary carcinoma, medulloblastoma, melanoma, meningioma, mesothelioma, myosarcoma, nasal cancer, neuroblastoma, oligodendroglioma, oral cancer, osteogenic sarcoma, ovarian cancer, pancreatic cancer, papillary adenocarcinoma, papillary carcinoma, pinealoma, prostate cancer, rhabdomyosarcoma, rectal cancer, renal cell carcinoma, retinoblastoma, sarcoma, sebaceous gland carcinoma, seminoma, skin cancer, squamous cell carcinoma, stomach cancer, sweat gland carcinoma, synovioma, testicular cancer, small cell lung carcinoma, throat cancer, uterine cancer, Wilm's tumor, blood cancer, acute erythroleukemic leukemia, acute lymphoblastic B-cell leukemia, acute lymphoblastic T-cell leukemia, acute lymphoblastic leukemia, acute megakaryoblastic leukemia, acute monoblastic leukemia, acute myeloblastic leukemia, acute myelomonocytic leukemia, acute nonlymphocytic leukemia, acute promyelocytic leukemia, acute undifferentiated leukemia, chronic lymphocytic leukemia, chronic myelocytic leukemia, hairy cell leukemia, multiple myeloma, heavy chain disease, Hodgkin's disease, multiple myeloma, non-Hodgkin's lymphoma, polycythemia vera, or Waldenstrom macroglobulinemia.

A crystalline phosphate salt provided by the present disclosure or a pharmaceutical composition thereof can be used to treat, for example, one or more of the following cancers: acute lymphoblastic leukemia, acute myeloid leukemia, adrenocortical carcinoma, appendix cancer, astrocytoma, atypical teratoid/rhabdoid tumor, basal cell carcinoma (nonmelanoma), B-cell lymphoma, bladder cancer, bone cancer, brain and spinal cord tumors, brain stem cancer, brain tumor, breast cancer, bronchial tumors, Burkitt lymphoma, carcinoid tumor, carcinoma of head and neck, central nervous system embryonal tumors, cerebellar astrocytoma, cerebral astrocytoma/malignant glioma, cervical cancer, chordoma, chronic lymphocytic leukemia, chronic myelogenous leukemia, colorectal cancer, craniopharyngioma, cutaneous T-cell lymphoma, desmoplastic small round cell tumor, ductal carcinoma, eye cancer, endocrine pancreas tumors (islet cell tumors), endometrial cancer, ependymoblastoma, esophageal cancer, esthesioneuroblastoma, Ewing family of tumors, extracranial germ cell tumor, extrahepatic bile duct cancer, gallbladder cancer, gastric cancer, gastrointestinal carcinoid tumor, gastrointestinal stromal tumor, gestational trophoblastic tumor, glioblastoma, glioma, hairy cell leukemia, head and neck cancer, heart cancer, hematopoetic tumors of the lymphoid lineage, hepatocellular cancer, Hodgkin lymphoma, hypopharyngeal cancer, hypothalamic and visual pathway glioma, IDs-related lymphoma, intraocular melanoma, islet cell tumors, Kaposi sarcoma, kidney cancer, Langerhans cell histiocytosis, laryngeal cancer, leukemia, lip and oral cavity cancer, male breast cancer, malignant fibrous histiocytoma, malignant germ cell tumors, malignant mesothelioma, medulloblastoma, melanoma, Merkel cell carcinoma, mesothelioma, mouth cancer, multiple endocrine neoplasia syndrome, multiple myeloma, mycosis fungoides, myelodysplastic, myeloproliferative neoplasms, nasal cavity and paranasal sinus cancer, nasopharyngeal cancer, neuroblastoma, non-Hodgkin lymphoma, non-small cell lung cancer, oral cancer, oropharyngeal cancer, osteosarcoma, ovarian cancer, ovarian epithelial cancer, ovarian germ cell tumor, ovarian low malignant potential tumor, pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pineal parenchymal tumors, pineoblastoma and supratentorial primitive neuroectodermal tumors, pituitary tumor, plasma cell neoplasm/multiple myeloma, pleuropulmonary blastoma, pregnancy and breast cancer, primary central nervous system lymphoma, primary liver cancer, primary metastatic squamous neck cancer with occult, prostate cancer, rectal cancer, renal cell cancer, renal pelvis and ureter, respiratory tract carcinoma, retinoblastoma, rhabdomyosarcoma, salivary gland cancer, sarcoma, Sézary syndrome, skin cancer, small intestine cancer, soft tissue sarcoma, squamous cell carcinoma (nonmelanoma), stomach cancer, supratentorial primitive neuroectodermal tumors, T-cell lymphoma, testicular cancer, throat cancer, thymoma and thymic carcinoma, thyroid cancer, transitional cell cancer, urethral cancer, uterine sarcoma, vaginal cancer, visual pathway and hypothalamic glioma, vulvar cancer, Waldenström macroglobulinemia, Wilms tumor, and systemic and central metastases of any of the foregoing.

Methods provided by the present disclosure include methods of treating cancer, where the cancer is selected from breast cancer and melanoma.

Methods provided by the present disclosure include methods of treating an inflammatory disease in a patient comprising administering to a patient in need of such treatment a therapeutically effective amount of a crystalline phosphate salt provided by the present disclosure or pharmaceutical composition thereof.

Examples of inflammatory diseases include allergy, Alzheimer's disease, anemia, ankylosing spondylitis, arthritis, atherosclerosis, asthma, autism, carpal tunnel syndrome, celiac disease, colitis, Crohn's disease, congestive heart failure, dermatitis, diabetes, diverticulitis, eczema, fibromyalgia, fibrosis, gall bladder disease, gastroesophageal reflux disease, Hashimoto's thyroiditis, heart attack, hepatitis, irritable bowel syndrome, kidney failure, lupus, multiple sclerosis, nephritis, neuropathy, pancreatitis, Parkinson's disease, psoriasis, polymyalgia rheumatica, rheumatoid arthritis, scleroderma, stroke, surgical complications, and ulcerative colitis.

Methods provided by the present disclosure include methods of treating an inflammatory disease in a patient, where the inflammatory disease is selected from, for example, acute respiratory distress syndrome, focal segmental glomerulonephritis, atherosclerosis/acute coronary syndrome, chronic obstructive pulmonary disease, asthma, inflammatory bowel disease, Crohn's disease, psoriasis, lupus, multiple sclerosis, inflammation in hypercholesterolemia, pain, diabetes, and rheumatoid arthritis.

Methods provided by the present disclosure include methods of treating an autoimmune disease in a patient comprising administering to a patient in need of such treatment a therapeutically effective amount of a crystalline phosphate salt provided by the present disclosure or pharmaceutical composition thereof.

A crystalline phosphate salt provided by the present disclosure, or a pharmaceutical composition thereof can be useful in treating autoimmune diseases. Autoimmune diseases can be defined as human diseases in which the immune system attacks its own proteins, cells, and/or tissues.

Examples of autoimmune diseases include Addison's disease, agammaglobulinemia, alopecia areata, amyloidosis, ankylosing spondylitis, anti-GBM/anti-TBN nephritis, antiphospholipid syndrome, autoimmune angioedema, autoimmune dysautonomia, autoimmune encephalomyelitis, autoimmune hepatitis, autoimmune inner ear disease, autoimmune myocarditis, autoimmune pancreatitis, autoimmune retinopathy, autoimmune urticaria, axonal and neuronal neuropathy, Balo disease, Behcet's disease, benign mucosal pemphigoid, bullous pemphigoid, Castleman disease, celiac disease, Chagas disease, chronic inflammatory demyelinating polyneuropathy, chronic recurrent multifocal osteomyelitis, Churg-Strauss, cicatricial pemphigoid, Cogan′ syndrome, cold agglutinin disease, congenital heart block, Coxsackie myocarditis, CREST syndrome, Crohn's disease, dermatitis herpetiformis, dermatomyositis, Devic's disease, discoid lupus, Dressler's syndrome, endometriosis, eosinophilic esophagitis, eosinophilic fasciitis, erythema nodosum, essential mixed cryoglobulinemia, Evans syndrome, fibromyalgia, fibrosing alveolitis, giant cell arteritis, giant cell myocarditis, glomerulonephritis, Goodpasture's syndrome, granulomatosis with polyangiitis, Graves' disease, Gullain-Barre syndrome, Hashimoto's thyroiditis, hemolytic anemia, Henoch-Schonlein purpura, herpes gestationis or pemphigoid gestationis, hypogammaglobulinemia, IgA nephropathy, IgG4-related sclerosing disease, immune thrombocytopeni purpura, inclusion body myositis, interstitial cystitis, juvenile arthritis, juvenile diabetes, juvenile myositis, Kawasaki disease, Lambert-Eaton syndrome, leukocytoclastic vasculitis, lichen planus, lichen sclerosis, ligneous conjunctivitis, linear IgA disease, lupus, Lyme disease chronic, Meniere's diseases, microscopic polyangiitis, mixed connective tissue disease, Mooren's ulcer, Mucha-Habermann disease, multiple sclerosis, myasthenia gravis, myositis, narcolepsy, neuromyelitis, optica, neutropenia, ocular cicatricial pemphigoid, optic neuritis, palindromic rheumatism, PANDAS, paraneoplastic cerebellar degeneration, paroxysmal nocturnal hemoglobinuria, Parry Romberg syndrome, pars planitis, Parsonnage-Turner syndrome, pemphigus, peripheral neuropathy, perivenous encephalomyelitis, pernicious anemia, POEMS syndrome, polyarteritis nodosa, polyglandular syndromes, polymyalgia rheumatica, polymyositis, post-myocardial infarction syndrome, post-pericardiotomy syndrome, primary biliary cirrhosis, primary sclerosing cholangitis, progesterone dermatitis, psoriasis, psoriatic arthritis, pure red cell aplasia, pyoderma gangrenosum, Raynaud's phenomenon, reactive arthritis, reflex sympathetic dystrophy, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm and testicular autoimmunity, stiff person syndrome, subacute bacterial endocarditis, Susac's syndrome, sympathetic ophthalmia, Takayasu's arteritis, temporal arteritis, thrombocytopenia purpura, Tolosa-Hunt syndrome, transverse myelitis, type 1 diabetes, ulcerative colitis, undifferentiated connective tissue disease, uveitis, vaculitis, vitiligo, and Wegener's granulomatosis.

A crystalline phosphate salt provided by the present disclosure, or a pharmaceutical composition thereof can be used to treat an autoimmune disease such as, for example, lupus, graft-versus-host disease, hepatitis C-induced vasculitis, Type I diabetes, multiple sclerosis, spontaneous loss of pregnancy, atopic diseases, and inflammatory bowel diseases.

A crystalline phosphate salt provided by the present disclosure, or a pharmaceutical composition thereof can be administered with one or more additional therapeutic agents for treating an autoimmune disease. A crystalline phosphate salt provided by the present disclosure or a pharmaceutical composition thereof may be administered in conjunction with one or more immunosuppressants including, for example, corticosteroids such as prednisone, budesonide, and prednisolone; Janus kinase inhibitors such as tofacitinib; calcineurin inhibitors such as cyclosporine and tacrolimus; mTOR inhibitors such as sirolimus and everolimus; IMDH inhibitors such as azathioprine, leflunomide, and mycophenolate; biologics such as abatacept adalimumab, anakinra, certolizumab, etanercept, golimumab, infliximab, ixekizumab, natalizumab, rituximab, secukinumab, tocilizumab, ustekinumab, and vedolizumab; and monoclonal antibodies such as basiliximab and daclizumab.

Methods provided by the present disclosure include methods of treating a disease in a patient comprising administering to a patient in need of such treatment a therapeutically effective amount of a crystalline phosphate salt provided by the present disclosure, wherein the disease is selected from acute coronary syndrome, acute lung injury, acute respiratory distress syndrome (ARDS), Alzheimer's disease, asthma, a cardiovascular disease, chronic obstructive pulmonary disease (COPD), inflammatory bowel disease, major depressive disorder, multiple sclerosis, neuropathic pain, and rheumatoid arthritis. A crystalline phosphate salt provided by the present disclosure, or a pharmaceutical composition thereof can be administered to a patient to treat a symptom of a viral infection such as a COVID19 infection.

Methods provided by the present disclosure include methods of treating a disease in a patient comprising administering to a patient in need of such treatment a therapeutically effective amount of a crystalline phosphate salt provided by the present disclosure, wherein the disease is a respiratory disease.

Methods provided by the present disclosure include methods of treating a disease in a patient comprising administering to a patient in need of such treatment a therapeutically effective amount of a crystalline phosphate salt provided by the present disclosure or pharmaceutical composition provided by the present disclosure, wherein the disease is an age-related disease such as, for example, hearing loss, muscle degeneration, Werner's syndrome, cellular aging, or Alzheimer's disease.

Methods provided by the present disclosure include methods of treating a disease in a patient comprising administering to a patient in need of such treatment a therapeutically effective amount of a crystalline phosphate salt provided by the present disclosure or a pharmaceutical composition of such treatment, wherein the disease is selected from sudden idiopathic hearing loss, drug induced hearing loss, age-related hearing loss, and Duchenne muscular dystrophy.

Methods provided by the present disclosure include methods of treating a disease in a patient comprising administering to a patient in need of such treatment a therapeutically effective amount of a crystalline phosphate salt provided by the present disclosure or a pharmaceutical composition of such treatment, wherein the disease is selected from a neuromuscular degeneration disease, Duchenne muscular dystrophy, amyotrophic lateral sclerosis, and a neurodegenerative disease.

Examples of neurodegenerative diseases include Alzheimer's disease, Parkinson's disease, amyotrophic lateral sclerosis, Huntington's disease, multiple sclerosis, prion diseases, frontotemporal dementia, Lewy body dementia, spinocerebellar ataxia, spinal muscular atrophy, progressive supranuclear palsy, corticobasal degeneration, chronic traumatic encephalopathy, vascular dementia, Kennedy's disease, Niemann-Pick disease, Gaucher's disease, Friedreich's ataxia, Wolfram syndrome, and Pelizaeus-Merzbacher disease.

Examples of neuromuscular degenerative diseases include amyotrophic lateral sclerosis, Duchenne muscular dystrophy, Becker muscular dystrophy, spinal muscular atrophy, Kennedy's disease, Charco-Mari-Tooth disease, myotonic dystrophy, Limb-Girdle muscular dystrophy, facioscapulohumeral muscular dystrophy, Lambert-Eaton myasthenic syndrome, myasthenia gravis, Friedreich's ataxia, hereditary spastic paraplegia, Pompe disease, Emery-Dreifuss muscular dystrophy, congenital muscular dystrophy, Bethlem myopathy, Ullrich congenital muscular dystrophy, nemaline myopathy, central core disease, multiminicore disease, inclusion body myositis, dermatomyositis, polymyositis, oculopharyngeal muscular dystrophy, distal muscular dystrophy, X-linked myotubular myopathy, rigid spine muscular dystrophy, Danon disease, and acid maltase deficiency.

The amount of a crystalline phosphate salt provided by the present disclosure, or pharmaceutical composition thereof that will be effective in the treatment of a cancer can depend, at least in part, on the nature of the disease, and may be determined by standard clinical techniques known in the art. In addition, in vitro or in vivo assays may be employed to help identify optimal dosing ranges. Dosing regimens and dosing intervals may also be determined by methods known to those skilled in the art. The amount of a crystalline phosphate salt provided by the present disclosure administered may depend on, among other factors, the patient being treated, the weight of the patient, the severity of the disease, the route of administration, and the judgment of the prescribing physician.

For systemic administration, a therapeutically effective dose may be estimated initially from in vitro assays. Initial doses may also be estimated from in vivo data, e.g., animal models, using techniques that are known in the art. Such information may be used to more accurately determine useful doses in humans. One having ordinary skill in the art may optimize administration to humans based on animal data.

A dose of a crystalline phosphate salt provided by the present disclosure and appropriate dosing intervals may be selected to maintain a sustained therapeutically effective concentration of 4-chloro-N-(4-(morpholinomethyl)phenyl)benzamide in the blood of a patient, and in certain embodiments, without exceeding a minimum adverse concentration.

A pharmaceutical composition comprising a crystalline phosphate salt provided by the present disclosure may be administered, for example, 4 times per day, twice a day, once a day, once per week, every 2 weeks, every 3 weeks, every 4 weeks, every 5 weeks, or every 6 weeks. Dosing may be provided alone or in combination with other drugs and may continue as long as required for effective treatment of the disease. Dosing may also be undertaken using continuous or semi-continuous administration over a period of time. Dosing includes administering a pharmaceutical composition to a mammal, such as a human, in a fed or fasted state.

A pharmaceutical composition may be administered in a single dosage form or in multiple dosage forms or as a continuous or an accumulated dose over a period of time. When multiple dosage forms are used the amount of a phosphate salt provided by the present disclosure contained within each of the multiple dosage forms may be the same or different.

Suitable daily dosage ranges for administration can range, for example, from about 2 μg to about 200 mg of a crystalline phosphate salt provided by the present disclosure per kilogram body weight.

Suitable daily dosage ranges for administration may range, for example, from about 1 μg to about 50 mg of crystalline phosphate salt provided by the present disclosure per square meter (m²) of body surface.

A crystalline phosphate salt provided by the present disclosure may be administered to treat a disease in a patient in an amount, for example, from 0.001 mg/day to 100 mg/day, or in any other appropriate daily dose. A dose can be, for example, from 0.01 μg/kg body weight/week to 100 μg/kg body weight/week or any other suitable dose.

A pharmaceutical composition comprising a crystalline phosphate salt provided by the present disclosure may be administered to treat a disease in a patient so as to provide a therapeutically effective concentration of 4-chloro-N-(4-(morpholinomethyl)phenyl)benzamide in the blood or plasma of the patient. A therapeutically effective concentration of 4-chloro-N-(4-(morpholinomethyl)phenyl)benzamide in the blood of a patient can be, for example, from 0.01 μg/L to 1,000 μg/L, from 0.1 μg/L to 500 μg/L, from 1 μg/L to 250 μg/L, or from about 10 μg/L to about 100 μg/L. A therapeutically effective concentration of 4-chloro-N-(4-(morpholinomethyl)phenyl)benzamide in the blood of a patient can be, for example, at least 0.01 μg/L, at least 0.1 μg/L, at least 1 μg/L, at least about 10 μg/L, or at least 100 μg/L. A therapeutically effective concentration of 4-chloro-N-(4-(morpholinomethyl)phenyl)benzamide in the blood of a patient can be, for example, less than an amount that causes unacceptable adverse effects including adverse effects to homeostasis. A therapeutically effective concentration of 4-chloro-N-(4-(morpholinomethyl)phenyl)benzamide in the blood of a patient can be an amount sufficient to restore and/or maintain homeostasis in the patient.

Pharmaceutical compositions provided by the present disclosure may be administered to treat a disease in a patient so as to provide a therapeutically effective concentration of 4-chloro-N-(4-(morpholinomethyl)phenyl)benzamide in the blood of a patient for a period of time such as, for example, for 4 hours, 8 hours, 12 hours, 16 hours, 20 hours, 1 day, or 2 days.

The amount of a crystalline phosphate salt provided by the present disclosure administered may vary during a treatment regimen.

Pharmaceutical compositions provided by the present disclosure may further comprise one or more pharmaceutically active compounds in addition to a crystalline phosphate salt provided by the present disclosure. Such compounds may be provided, for example, to treat the disease being treated with a crystalline phosphate salt provided by the present disclosure or to treat a disease, disorder, or condition other than the disease being treated with a phosphate salt provided by the present disclosure, to treat a side-effect caused by 4-chloro-N-(4-(morpholinomethyl)phenyl)benzamide to augment the efficacy of 4-chloro-N-(4-(morpholinomethyl)phenyl)benzamide and/or to modulate the activity of 4-chloro-N-(4-(morpholinomethyl)phenyl)benzamide.

A crystalline phosphate salt provided by the present disclosure may be administered in combination with at least one other therapeutic agent. A crystalline phosphate salt provided by the present disclosure may be administered to a patient together with another compound for treating the disease in the patient. A crystalline phosphate salt provided by the present disclosure and the at least one other therapeutic agent may act additively or, and in certain embodiments, synergistically. The at least one additional therapeutic agent may be included in the same pharmaceutical composition or vehicle comprising a phosphate salt provided by the present disclosure or may be in a separate pharmaceutical composition or vehicle. Accordingly, methods provided by the present disclosure further include, in addition to administering a crystalline phosphate salt provided by the present disclosure, administering one or more therapeutic agents effective for treating cancer or a different disease, disorder or condition than cancer. Methods provided by the present disclosure include administering a crystalline phosphate salt provided by the present disclosure and one or more other therapeutic agents provided that the combined administration does not inhibit the therapeutic efficacy of 4-chloro-N-(4-(morpholinomethyl)phenyl)benzamide and/or does not produce significant adverse combination effects.

A pharmaceutical composition comprising a crystalline phosphate salt provided by the present disclosure may be administered concurrently with the administration of another therapeutic agent, which may be part of the same pharmaceutical composition as, or in a different pharmaceutical composition than that comprising a crystalline phosphate salt provided by the present disclosure. A crystalline phosphate salt provided by the present disclosure may be administered prior or subsequent to administration of another therapeutic agent. In certain combination therapies, the combination therapy may comprise alternating between administering a crystalline phosphate salt provided by the present disclosure and a composition comprising another therapeutic agent, e.g., to minimize adverse drug effects associated with a particular drug. When a crystalline phosphate salt provided by the present disclosure is administered concurrently with another therapeutic agent that potentially may produce an adverse drug effect including, for example, toxicity, the other therapeutic agent may be administered at a dose that falls below the threshold at which the adverse drug reaction is elicited.

A pharmaceutical composition comprising a phosphate salt provided by the present disclosure may be administered with one or more substances, for example, to enhance, modulate and/or control release, bioavailability, therapeutic efficacy, therapeutic potency, and/or stability, of a crystalline phosphate salt provided by the present disclosure. For example, a pharmaceutical composition comprising a phosphate salt provided by the present disclosure can be co-administered with an active agent having pharmacological effects that enhance the therapeutic efficacy of 4-chloro-N-(4-(morpholinomethyl)phenyl)benzamide.

A crystalline phosphate salt provided by the present disclosure, or a pharmaceutical composition thereof may be administered in conjunction with an agent known or believed to be effective in treating a disease such as cancer, an autoimmune disease, an inflammatory disease, or an age-related disease in a patient, such as the same disease being treated with a phosphate salt provided by the present disclosure.

The efficacy of administering a crystalline phosphate salt provided by the present disclosure or a pharmaceutical composition thereof for treating cancer, an inflammatory disease, an autoimmune disease, or an age-related disease may be assessed using in vitro and animal studies and in clinical trials.

### ASPECTS OF THE INVENTION

The invention is further defined by one or more of the following aspects.

Aspect 1. Crystalline 4-chloro-N-(4-(morpholinomethyl)phenyl)benzamide phosphate salt Form 4A.

Aspect 2. The crystalline phosphate salt of aspect 1, wherein the crystalline phosphate salt Form 4A is characterized by an XRPD pattern comprising characteristic diffraction peaks at least at 7.9°±0.2°, 13.7°±0.2°, 16.8°±0.2°, 17.8°±0.2°, 18.6°±0.2°, 21.3°±0.2°, 23.5°±0.2°, 23.9°±0.2°, and 26.1°±0.2°, expressed as θ/2θ angles and determined using Cu-Kα radiation.

Aspect 3. The crystalline phosphate salt of aspect 1, wherein the crystalline phosphate salt Form 4A is characterized by an XRPD pattern comprising characteristic diffraction peaks at least at 7.9°±0.1°, 13.7°±0.1°, 16.8°±0.1°, 17.8°±0.1°, 18.6°±0.1°, 21.3°±0.1°, 23.5°±0.1°, 23.9°±0.1°, and 26.1°±0.1°, expressed as θ/2θ angles and determined using Cu-Kα radiation.

Aspect 4. The crystalline phosphate salt of aspect 1, wherein the crystalline phosphate salt Form 4A is characterized by an XRPD pattern comprising characteristic diffraction peaks at least at 7.9°±0.2°, 8.4°±0.2°, 13.7°±0.2°, 16.8°±0.2°, 17.0°±0.2°, 17.8°±0.2°, 18.6°±0.2°, 21.3°±0.2°, 21.7°±0.2°, 23.5°±0.2°, 23.9°±0.2°, 24.1°±0.2°, and 26.1°±0.2°, expressed as θ/2θ angles and determined using Cu-Kα radiation.

Aspect 5. The crystalline phosphate salt of aspect 1, wherein the crystalline phosphate salt Form 4A is characterized by an XRPD pattern comprising characteristic diffraction peaks at least at 7.9°±0.1°, 8.4°±0.1°, 13.7°±0.1°, 16.8°±0.1°, 17.0°±0.1°, 17.8°±0.1°, 18.6°±0.1°, 21.3°±0.1°, 21.7°±0.1°, 23.5°±0.1°, 23.9°±0.1°, 24.1°±0.1°, and 26.1°±0.1°, expressed as θ/2θ angles and determined using Cu-Kα radiation.

Aspect 6. The crystalline phosphate salt of aspect 1, wherein the crystalline phosphate salt Form 4A is characterized by an XRPD pattern substantially as shown in FIG. 2.

Aspect 7. The crystalline phosphate salt of aspect 1, wherein the crystalline phosphate salt Form 4A is characterized by an XRPD pattern substantially as shown in FIG. 3.

Aspect 8. The crystalline phosphate salt of any one of aspects 1 to 7, wherein the crystalline phosphate salt Form 4A has a differential scanning calorimetry curve characterized by a sharp endotherm with an onset temperature from 219° C. to 230° C.

Aspect 9. The crystalline phosphate salt of any one of aspects 1 to 8, wherein the crystalline phosphate salt Form 4A has a differential scanning calorimetry curve characterized by an exothermic peak from 229° C. to 232° C.

Aspect 10. The crystalline phosphate salt of any one of aspects 1 to 9, wherein the crystalline phosphate salt Form 4A has a differential scanning calorimetry curve characterized by an exothermic enthalpy from 137 J/g to 147 J/g.

Aspect 11. The crystalline phosphate salt of any one of aspects 1 to 10, wherein the crystalline phosphate salt Form 4A has a differential scanning calorimetry curve characterized by a broad endotherm with an onset temperature from 165° C. to 174° C.

Aspect 12. The crystalline phosphate salt of any one of aspects 1 to 11, wherein the crystalline phosphate salt Form 4A has a differential scanning calorimetry curve characterized by an endothermic peak from 170° C. to 180° C.

Aspect 13. The crystalline phosphate salt of any one of aspects 1 to 12, wherein the crystalline phosphate salt Form 4A has a differential scanning calorimetry curve characterized by an endothermic enthalpy from 1 J/g to 3 J/g.

Aspect 14. The crystalline phosphate salt of any one of aspects 1 to 7, wherein the crystalline phosphate salt Form 4A has a differential scanning calorimetry curve substantially as shown in any one of FIGS. 4-7.

Aspect 15. The crystalline phosphate salt of any one of aspects 1 to 14, wherein the crystalline phosphate salt Form 4A has a thermogravimetric analysis curve characterized by a weight loss of from 0% to 0.25% within a temperature range from 30° C. to 150° C., and with decomposition at higher temperatures.

Aspect 16. The crystalline phosphate salt of any one of aspects 1 to 14, wherein the crystalline phosphate salt Form 4A has a thermogravimetric analysis curve substantially as shown in any one of FIGS. 4-6.

Aspect 17. The crystalline phosphate salt of any one of aspects 1 to 16, wherein the crystalline phosphate salt exhibits a weight gain of less than 2.5% from 0% RH to 95% RH as determined by dynamic vapor sorption.

Aspect 18. The crystalline phosphate salt of any one of aspects 1 to 17, wherein the crystalline phosphate salt has a solubility in a 1:1 ACN:water mixture from 50 mg/mL to 60 mg/mL at 40° C.

Aspect 19. The crystalline phosphate salt of any one of aspects 1 to 18, wherein the crystalline phosphate salt has a solubility in a 1:1 THF:water mixture from 75 mg/mL to 85 mg/mL at 40° C.

Aspect 20. The crystalline phosphate salt of any one of aspects 1 to 19, wherein the crystalline phosphate salt is stable following exposure to 40° C./75% RH for at least one week.

Aspect 21. The crystalline phosphate salt of any one of aspects 1 to 20, wherein the crystalline phosphate salt has a purity greater than 95%.

Aspect 22. The crystalline phosphate salt of any one of aspects 1 to 20, wherein the crystalline phosphate salt has a purity greater than 98%.

Aspect 23. Crystalline 4-chloro-N-(4-(morpholinomethyl)phenyl)benzamide phosphate salt Form 4C.

Aspect 24. The crystalline phosphate salt of aspect 23, wherein the crystalline phosphate salt Form 4C is characterized by an XRPD pattern comprising characteristic diffraction peaks at least at 13.7°±0.2°, 17.3°±0.2°, 19.8°±0.2°, 20.5°±0.2°, 22.6°±0.2°, and 25.1°±0.2°, expressed as θ/2θ angles and determined using Cu-Kα radiation.

Aspect 25. The crystalline phosphate salt of aspect 23, wherein the crystalline phosphate salt Form 4C is characterized by an XRPD pattern comprising characteristic diffraction peaks at least at 13.7°±0.1°, 17.3°±0.1°, 19.8°±0.1°, 20.5°±0.1°, 22.6°±0.1°, and 25.1°±0.1°, expressed as θ/2θ angles and determined using Cu-Kα radiation.

Aspect 26. The crystalline phosphate salt of aspect 23, wherein the crystalline phosphate salt Form 4C is characterized by an XRPD pattern comprising characteristic diffraction peaks at least at 13.5°±0.2°, 13.7°±0.2°, 15.9°±0.2°, 17.3°±0.2°, 19.5°±0.2°, 19.8°±0.2°, 20.5°±0.2°, 22.6°±0.2°, 24.0°±0.2°, 24.4°±0.2°, and 25.1°±0.2°, expressed as θ/2θ angles and determined using Cu-Kα radiation.

Aspect 27. The crystalline phosphate salt of aspect 23, wherein the crystalline phosphate salt Form 4C is characterized by an XRPD pattern comprising characteristic diffraction peaks at least at 13.5°±0.1°, 13.7°±0.1°, 15.9°±0.1°, 17.3°±0.1°, 19.5°±0.1°, 19.8°±0.1°, 20.5°±0.1°, 22.6°±0.1°, 24.0°±0.1°, 24.4°±0.1°, and 25.1°±0.1°, expressed as θ/2θ angles and determined using Cu-Kα radiation.

Aspect 28. The crystalline phosphate salt of aspect 23, wherein the crystalline phosphate salt Form 4C is characterized by an XRPD pattern substantially as shown in FIG. 9.

Aspect 29. The crystalline phosphate salt of any one of aspects 23 to 28, wherein the crystalline phosphate salt Form 4C is characterized by an DSC profile having a small endotherm with an onset temperature at 34.1° C.±5° C. or 34.1° C.±2.0° C., followed by an endotherm with an onset temperature at 153.4° C.±5.0° C. or 153.4° C.±2.0° C. with a peak temperature at 172.0° C.±5.0° C. or 172.0° C.±2.0° C.

Aspect 30. The crystalline phosphate salt of any one of aspects 23 to 28, wherein the crystalline phosphate salt Form 4C is characterized by a DSC profile substantially as shown in FIG. 10.

Aspect 31. The crystalline phosphate salt of any one of aspects 23 to 30, wherein the crystalline phosphate salt Form 4C is characterized by a TGA profile having a weight loss of from 0.5% to 2.0%, such as from 1.0% to 0.15%, or about 1.35% within a temperature range from 30° C. to 180° C., and with decomposition at higher temperatures.

Aspect 32. The crystalline phosphate salt of any one of aspects 23 to 30, wherein the crystalline phosphate salt Form 4C is characterized by a TGA profile substantially as shown in FIG. 10.

Aspect 33. The crystalline phosphate salt of any one of aspects 23 to 32, wherein the crystalline phosphate salt Form 4C is characterized by a purity, for example, greater than 90 wt %, greater than 92 wt %, greater than 94 wt %, greater than 96 wt %, greater than 98 wt %, greater than 99 wt %, greater than 99.5 wt %, or greater than 99.9 wt %, where wt % is based on the total weight of the crystalline 4-chloro-N-(4-(morpholinomethyl)phenyl)benzamide phosphate Form 4C.

Aspect 34. A pharmaceutical composition comprising the crystalline phosphate salt of any one of aspects 1 to 33 and a pharmaceutically acceptable vehicle.

Aspect 35. The pharmaceutical composition of aspect 34, wherein the pharmaceutical composition is an oral pharmaceutical composition.

Aspect 36. The pharmaceutical composition of aspect 34, wherein the pharmaceutical composition is an oral dosage form.

Aspect 37. The pharmaceutical composition of any one of aspects 34 to 36, wherein the pharmaceutical composition comprises a therapeutically effective amount of the phosphate salt for treating a disease in a patient, wherein the disease is selected from cancer, an inflammatory disease, an autoimmune disease, an age-related disease, and a respiratory disease.

Aspect 38. The pharmaceutical composition of aspect 37, wherein the disease is cancer.

Aspect 39. The pharmaceutical composition of aspect 38, wherein the cancer is selected from breast cancer and melanoma.

Aspect 40. The pharmaceutical composition of aspect 37, wherein the disease is an inflammatory disease.

Aspect 41. The pharmaceutical composition of aspect 40, wherein the inflammatory disease is selected from acute respiratory distress syndrome, focal segmental glomerulonephritis, atherosclerosis/acute coronary syndrome, chronic obstructive pulmonary disease, asthma, inflammatory bowel disease, Crohn's disease, psoriasis, lupus, multiple sclerosis, inflammation in hypercholesteremia, pain, diabetes, and rheumatoid arthritis.

Aspect 42. The pharmaceutical composition of aspect 40, wherein the inflammatory disease is selected from acute lung injury, acute respiratory distress syndrome (ARDS), and chronic obstructive pulmonary disease (COPD).

Aspect 43. The pharmaceutical composition of aspect 39, wherein the disease is an autoimmune disease.

Aspect 44. The pharmaceutical composition of aspect 39, wherein the disease is an age-related disease.

Aspect 45. The pharmaceutical composition of aspect 44, wherein the age-related disease is selected from hearing loss, muscle degeneration, Werner's syndrome, cellular aging, and Alzheimer's disease.

Aspect 46. A method of treating a disease in a patient comprising administering to a patient in need of such treatment a therapeutically effective amount of the crystalline phosphate salt of any one of aspects 1 to 33 or the pharmaceutical composition of any one of aspects 34 to 37, wherein the disease is selected from cancer, an inflammatory disease, an autoimmune disease, a respiratory disease, and an age-related disease.

Aspect 47. The method of aspect 46, wherein the disease is cancer.

Aspect 48. The method of aspect 47, wherein the cancer is selected from breast cancer and melanoma.

Aspect 49. The method of aspect 46, wherein the disease is an inflammatory disease.

Aspect 50. The method of aspect 49, wherein the inflammatory disease is selected from acute respiratory distress syndrome, focal segmental glomerulonephritis, atherosclerosis/acute coronary syndrome, chronic obstructive pulmonary disease, asthma, inflammatory bowel disease, Crohn's disease, psoriasis, lupus, multiple sclerosis, inflammation in hypercholesteremia, pain, diabetes, and rheumatoid arthritis.

Aspect 51. The method of aspect 49, wherein the inflammatory disease is selected from acute lung injury, acute respiratory distress syndrome (ARDS), and chronic obstructive pulmonary disease (COPD).

Aspect 52. The method of aspect 46, wherein the disease is an autoimmune disease.

Aspect 53. The method of aspect 46, wherein the disease is an age-related disease.

Aspect 54. The method of aspect 53, wherein the age-related disease is selected from hearing loss, muscle degeneration, Werner's syndrome, cellular aging, and Alzheimer's disease.

Aspect 55. A method of treating a disease in a patient comprising administering to a patient in need of such treatment a therapeutically effective amount of the crystalline phosphate salt of any one of aspects 1 to 33 or the pharmaceutical composition of any one of aspects 34 to 36, wherein the disease is selected from acute lung injury, acute respiratory distress syndrome (ARDS), and chronic obstructive pulmonary disease (COPD).

Aspect 56. A method of treating a disease in a patient comprising administering to a patient in need of such treatment a therapeutically effective amount of the crystalline phosphate salt of any one of aspects 1 to 33 or the pharmaceutical composition of any one of aspects 34 to 36, wherein the disease is treated by modulating the p38α MAPK receptor.

Aspect 57. A method of modulating the p38α MAPK receptor in a patient comprising administering to a patient a pharmacologically effective amount of the crystalline phosphate salt of any one of aspects 1 to 33 or the pharmaceutical composition of any one of aspects 34 to 36.

Aspect 58. The method of aspect 57, wherein modulating the p38α MAPK receptor comprises selectively inhibiting the p38α MAPK receptor.

Aspect 59. The method of any one of aspects 57 to 58, wherein modulating the p38α MAPK receptor does not result in loss of a p38α-dependent counterregulatory response.

Aspect 60. The method of aspect 59, wherein the p38α-dependent counterregulatory response relates to mitogen- and stress-activated protein kinase-1 (MSK1) or MSK2.

Aspect 61. The method of any one of aspects 56 to 60, wherein modulating the p38α MAPK receptor stabilizes an endothelial or epithelial barrier function.

Aspect 62. The method of any one of aspects 56 to 61, wherein modulating the p38α MAPK receptor reduces inflammation.

Aspect 63. The method of any one of aspects 56 to 62, wherein modulating the p38α MAPK receptor mitigates LPS-induced lung injury.

Aspect 64. The method of any one of aspects 56 to 63, wherein modulating the p38α MAPK receptor regulates leukocyte trafficking.

Aspect 65. The method of any one of aspects 56 to 64, wherein modulating the p38α MAPK receptor regulates cytokine expression.

Aspect 66. A kit comprising the crystalline phosphate salt of any one of aspects 1 to 33 or the pharmaceutical composition of any one of aspects 34 to 36.

Aspect 67. A method of treating a disease in a patient comprising administering to a patient in need of such treatment a therapeutically effective amount of the crystalline phosphate salt of any one of aspects 1 to 33 or the pharmaceutical composition of any one of aspects 34 to 36, wherein the disease is selected from a neurodegenerative disease and a neuromuscular degenerative disease.

Aspect 68. A method of treating a disease in a patient comprising administering to a patient in need of such treatment a therapeutically effective amount of the crystalline phosphate salt of any one of aspects 1 to 33 or the pharmaceutical composition of any one of aspects 34 to 36, wherein the disease is selected from amyotrophic lateral sclerosis and Duchenne muscular dystrophy.

### EXAMPLES

The following examples describe in detail methods of preparing crystalline phosphate salts of 4-chloro-N-(4-(morpholinomethyl)phenyl)benzamide, properties of the crystalline phosphate salts, and methods of using the crystalline phosphate salts. It will be apparent to those skilled in the art that many modifications, both to materials and methods, may be practiced without departing from the scope of the invention.

### Instrumentation

### X-ray Powder Diffraction

X-ray Powder Diffraction (XRPD) patterns were collected on a Bruker D4 Endeavor® instrument. The instrument and method details are included in Table 1.

TABLE 1
Bruker D4 Endeavor Instrument Parameters.

| Parameter | Bruker D4 Endeavor | |
|---|---|---|
| Sample Holder | PMMA Sample Holder with Zero | |
| | Background Silicon Wafer | |
| Scan Range | 3° to 40° 2θ | |
| Scan Type | Coupled 2θ/θ | |
| Scan Mode | Continuous | |
| Step Size | 0.003°-0.004° | |
| Time/Step | 0.0357-0.2724 | seconds |
| Wavelength | Cu Kα 1.54 Å | |
| X-ray Generator | 40 kV/40 mA | |
| Detector | LynxEye | |
| Detector Slit | 8.0 | mm |
| Divergence Slit | 2.0 | mm |
| Anti-Scatter Slit | 6.21 | mm |

Differential Scanning calorimetry

Differential scanning calorimetry (DSC) thermograms were collected on a TA Instruments DSC 25 equipped with a Refrigerated Cooling System. The instrument and method details are summarized in Table 2.

TABLE 2
TA DSC 25 Instrument Parameters.

| Parameters | TA DSC 25 with Refrigerated Cooling System |
|---|---|
| Sample Holder | TA Tzero Aluminum Pans and Lids |
| Scan Temp Range | 30 °C to 330 °C |
| Scan Rate | 10 °C/min |
| Purge | Nitrogen, 50 cc/min |

### Thermogravimetric Analysis

Thermogravimetric Analysis (TGA) thermograms were collected on a Perkin Elmer TGA-7 Instrument. The instrument parameters are summarized in Table 3.

TABLE 3
Perkin Elmer TGA-7 Instrument and Method Details.

| Parameters | Perkin Elmer TGA-7 |
|---|---|
| Sample Holder | Mettler Toledo Al Crucibles 40 μL |
| Scan Temp Range | 30 °C to 330 °C |
| Scan Rate | 10 °C/min |
| Purge | Nitrogen, 20 cc/min |

### Dynamic Vapor Sorption

Dynamic Vapor Sorption (DVS) plots were collected on a Surface Measurement Systems DVS Intrinsic PLUS®. The instrument parameters are summarized in Table 4.

TABLE 4
Surface Measurement Systems DVS Intrinsic
Plus Instrument Parameters.

| | Surface Measurement Systems |
|---|---|
| Parameters | DVS Intrinsic PLUS ® |
| Sample Holder | 10 mm Metal Pan |
| Temperature | 25 °C |
| % RH Cycles | 50% RH to 0% RH to 95% RH to 50% RH |
| Purge | Nitrogen, 200 cc/min |

### Water Content

Water content of samples was measured on a Mettler Toledo C10S Coulometric KF Titrator. The instrument parameters are provided in Table 5.

TABLE 5
Mettler Toledo C10S Coulometric KF
Titrator Instrument Parameters.

| Parameters | Mettler Toledo C10S Coulometric KF Titrator | |
|---|---|---|
| Generator current | Automatic | |
| (w/out diaphragm) | | |
| Weight Range | 0.0-5.0 | g |
| KF Reagent | CombiCoulomat fritless | |
| I (pol) | 5.0 | μA |
| Start drift | <25 | μg/min |
| Mix time | 60 | seconds |
| End Point at | 100.0 | mV |
| Dynamics | 250.0 | mV |
| Stop criterion | Drift stop relative | |
| Stop drift | 3.0 | μg/min |

### Example 1

### Synthesis of 4-Chloro-N-(4-(morpholinomethyl)phenyl)benzamide

4-Chloro-N-(4-(morpholinomethyl)phenyl)benzamide was prepared by reacting 4-chlorobenzoyl chloride with 4-(morpholinomethyl) aniline in an inert solvent such as dichloromethane (DCM), triethylamine (TEA) or diethanolamine (DEA) in the presence of a nucleophilic catalyst such as 4-(dimethylamino)pyridine (DMAP) at a temperature, for example, from 20° C. to 60° C.

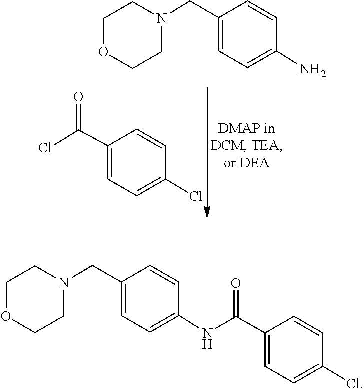 [SMILES: Nc1ccc(CN2CCOCC2)cc1 ; O=C(Cl)c1ccc(Cl)cc1 ; O=C(Nc1ccc(CN2CCOCC2)cc1)c1ccc(Cl)cc1]

### Example 2

### Preparation of Crystalline 4-Chloro-N-(4-(morpholinomethyl)phenyl)benzamide

### Phosphate Salt Form 4A

To prepare crystalline phosphate salt Form 4A, 500 mg 4-chloro-N-(4-(morpholinomethyl)phenyl)benzamide, 10 volumes of tetrahydrofuran (THF) and a 1.1:1 molar ratio of phosphoric acid were combined and stirred at 40° C., to provide a yellow gel suspended in a cloudy solution after 4 hours stirring and an off-white slurry after overnight stirring. A 1 mL sample was centrifuged to recover the solids, which were analyzed by XRPD. The remaining slurry was vacuum filtered, and placed in a vacuum oven at 40° C. to obtain 591.0 mg of crystalline phosphate salt Form 4A (salt yield ~91.2%).

For scale-up preparation of the crystalline phosphate salt Form 4A, 1.5 g of the free base was combined with 5 volumes of THF and stirred at 40° C. About 7.841 mL of a diluted counterion stock solution (1.1:1 molar ratio phosphoric acid and 5 vols. THF) was added in 5 steps every 30 minutes and seeded with about 19.4 mg (~1% salt) crystalline phosphate salt Form 4A seeds to provide an off-white slurry after overnight stirring.

About 1.5 mL of the slurry was sampled and centrifuged to recover the solids. The recovered solids were analyzed by XRPD. The remaining slurry was vacuum filtered, dried in a vacuum oven at 40° C. to provide about 1.7 g (87.4% yield) of crystalline phosphate salt Form 4A.

In another scale-up preparation 2.5 g of the free base, 5 volumes of THE, were stirred at 40° C., and 13.068 mL of a diluted phosphoric acid solution (1.1:1 molar ratio counterion+5 vols. solvent) was added in 5 steps every 30 minutes. The mixture was then seeded with 32.4 mg (~1% salt) of crystalline phosphate salt Form 4A seeds to provide an off-white slurry after overnight stirring. The slurry was sampled (1 mL) and centrifuged to recover the solids, which were analyzed by XRPD. The remaining slurry was vacuum-filtered and placed in a vacuum oven at 40° C. to provide 3.19 g of crystalline phosphate salt Form 4A (98% yield) as an off-white powder. TGA data confirmed a low total volatile content of 0.09%, no additional testing was completed.

### Example 3

### Polymorph and Hydrate Screening 4-Chloro-N-(4-(morpholinomethyl)phenyl)benzamide Phosphate Salt

Eighty-two (82) total solid form screening experiments were performed to identify solid forms of crystalline 4-chloro-N-(4-(morpholinomethyl)phenyl)benzamide phosphate salt.

Seventeen slurry experiments were undertaken. Forty (40) mg of the crystalline phosphate salt Form 4A was added to 20 volumes of solvent, stirred at 23 C overnight to provide an off-white slurry after overnight stirring. The slurry was centrifuged to recover solids, analyzed by XRPD, and the remainder placed in a vacuum oven at 40° C. The following solvents were used: acetone, methanol, ethanol, isopropyl alcohol, 1-propanol, MTBE, THF, CAN, DCM, DMF, ethyl acetate, heptane, water, acetone:water (90:10), ethanol:water (90:10), DCM:methanol (1:1), and ACN:water (1:1).

Two evaporation experiments were undertaken. Forty (40) mg of crystalline phosphate salt Form 4A was added to 14 volumes solvent (either DMSO or THF:water (1:1)) and stirred at 23 C to provide a clear solution. The solution was opened for evaporation to provide a thin off-white slurry after overnight evaporation. The slurry was centrifuged to recover the solids, analyzed by XRPD and dried in a vacuum oven at 40° C.

Ten anti-solvent vapor diffusion experiments at room temperature were performed. Forty (40) mg of crystalline phosphate salt Form 4A was added 14.5 vols. of solvent, stirred at 23 C to dissolve the solids. The contents were added to a 20 mL vial containing 8 mL anti-solvent and the closed and sealed with Parafilm®. After 7 days of diffusing an off-white solid resulted. The solvent was removed to isolate the solids, which were analyzed by XRPD, and placed in the vacuum oven at 40° C. to dry. The following antisolvents were used with the solvent DMSO:acetone, ACN, DCM, ethanol, ethyl acetate, isopropyl alcohol, MTBE, THF, and water. The antisolvent THF was used with the solvent THF:water (1:1).

For slow room temperature anti-solvent experiments 40 mg crystalline phosphate salt Form 4A was dissolved in 14 vols. solvent at RT. One (1) vol. solvent was added to undersaturation, followed by the addition of 30 vols. anti-solvent at a rate of 2.14 vol./30 min to provide a clear solution after overnight stirring. The solvent was evaporated for from 2 hours to 10 days to provide a clear solution with an off-white solid. The solvent was pipetted to isolate the solids, which were analyzed by PXRD, and the remainder dried in a vacuum oven at 40° C. DMSO:acetone, DMSO:DMF, DMSO: ethanol, or THF:water (1:1):water were used as the solvent/anti-solvent combinations.

For fast room temperature anti-solvent experiments, 40 mg crystalline phosphate salt Form 4A was dissolved in 14 vols. solvent at RT. One (1) vol. solvent was added to undersaturation, followed by the addition of 30 vols. anti-solvent at a rate of 2.14 vol./30 min to provide a clear solution after overnight stirring. The solvent was evaporated for from 2 hours to 10 days to provide a clear solution with an off-white solid. The solvent was pipetted to isolate the solids, which were analyzed by XRPD, and the remainder dried in a vacuum oven at 40° C. DMSO:acetone, DMSO:DMF, DMSO: ethanol, or THF:water (1:1):water were used as the solvent/anti-solvent combinations.

For reactive crystallization experiments, 40 mg of crystalline phosphate salt Form 4A was dissolved in 10 vols. solvent and a 1.1:1 molar ratio phosphoric acid was added and the mixture, stirred at 40° C. to provide a clear solution. The solvent was evaporated overnight to provide an off-white slurry after overnight. The slurry was centrifuged to recover the solids, which were analyzed by PXRD, and the remainder dried in a vacuum oven at 40° C. The following solvents were used: water, ACN:water (1:1), ethanol:water (1:1), DMSO, DMSO, DMSO: water (1:1), DMSO:DCM (1:1), and THF:water (1:1).

For grinding experiments 40 mg crystalline phosphate salt 4A and 0.5 volume solvent was ground in ball mill at 3000 rpm for 15 seconds and then analyzed by XRPD, and dried in a vacuum oven at 40° C. The following solvents were used: no solvent, ACN, DCM, DMSO, ethyl acetate, ethanol, heptane, water, THF, and THE: water (1:1).

For DSC recrystallization experiments, from about 10 mg to about 13 mg of crystalline phosphate salt 4A in DSC pan, heated from 30° C. to 190° C. at 10° C./min, cooled from 190° C. to 30° C. at 10° C./min

For slurry experiments at 40° C., forty (40) mg of crystalline phosphate salt 4A and 20 vols. solvent were stirred overnight at 40° C. to provide an off-white slurry. The slurry was centrifuged to recover the solids, analyzed by XRPD, and placed in the vacuum oven at 40° C. to dry. The following solvents were used: acetone, ethanol, isopropyl alcohol, MTBE, ACN, ethyl acetate, heptane, and water.

For cooling/recrystallization experiments, 40 mg of crystalline phosphate salt 4A and 14 vols. solvent (ACN:water (1:1) or THF:water (1:1)) were stirred at 40° C. to provide a clear solution. After removing the heat and overnight stirring, a clear solution was provided. The solution was allowed to evaporate to provide an off-white slurry after overnight stirring. The slurry was centrifuged to recover solids, analyzed by XRPD, and dried in the vacuum oven at 40° C.

Two stable crystalline forms were observed: Form 4A and Form 4C.

### Example 4

### Optical Microscopy of Crystalline 4-Chloro-N-(4-(morpholinomethyl)phenyl)benzamide

### Phosphate Salt Form 4A

An optical micrograph of the crystalline phosphate salt form 4A is shown in FIG. 1. The material had a morphology characterized by granular-like, irregular agglomerates, and fine particles.

### Example 5

### XRPD of Crystalline 4-Chloro-N-(4-(morpholinomethyl)phenyl)benzamide

### Phosphate Salt Form 4A

FIG. 2 and FIG. 3 show XRPD patterns of the crystalline phosphate salt Form 4A. The diffraction peak listings for the XRPD shown in FIG. 2 are provided in Table 6.

TABLE 6
Crystalline phosphate Salt Form 4A XRPD Peak Listing.

| Angle (°2θ) | Intensity | Relative Intensity |
|---|---|---|
| 7.89 | 594.24 | 54% |
| 8.44 | 137.95 | 12% |
| 12.11 | 20.10 | 2% |
| 13.30 | 37.61 | 3% |
| 13.72 | 185.95 | 17% |
| 14.99 | 30.44 | 3% |
| 15.65 | 74.33 | 7% |
| 15.86 | 61.93 | 6% |
| 16.20 | 124.38 | 11% |
| 16.33 | 104.81 | 9% |
| 16.85 | 360.61 | 33% |
| 16.96 | 300.43 | 27% |
| 17.15 | 58.69 | 5% |
| 17.80 | 1104.31 | 100% |
| 18.56 | 436.30 | 40% |
| 19.31 | 76.94 | 7% |
| 19.94 | 66.87 | 6% |
| 21.34 | 222.59 | 20% |
| 21.73 | 199.07 | 18% |
| 22.24 | 49.86 | 5% |
| 22.58 | 35.74 | 3% |
| 23.28 | 101.75 | 9% |
| 23.52 | 749.88 | 68% |
| 23.87 | 882.71 | 80% |
| 24.11 | 389.60 | 35% |
| 24.83 | 136.01 | 12% |
| 25.86 | 185.43 | 17% |
| 26.13 | 380.53 | 34% |
| 26.71 | 192.06 | 17% |
| 27.07 | 200.63 | 18% |
| 27.61 | 79.83 | 7% |
| 28.40 | 36.47 | 3% |
| 28.68 | 45.85 | 4% |
| 29.08 | 27.74 | 3% |
| 30.38 | 115.45 | 10% |
| 31.47 | 35.55 | 3% |
| 32.25 | 55.59 | 5% |
| 32.72 | 36.57 | 3% |
| 33.53 | 62.72 | 6% |
| 33.93 | 95.57 | 9% |
| 34.58 | 34.31 | 3% |
| 35.08 | 64.28 | 6% |
| 35.85 | 51.09 | 5% |
| 37.04 | 31.33 | 3% |
| 37.54 | 28.41 | 3% |
| 38.69 | 33.83 | 3% |

### Example 6

### DSC/TGA of Crystalline 4-Chloro-N-(4-(morpholinomethyl)phenyl)benzamide

### Phosphate Salt Form 4A

DSC thermograms of the crystalline phosphate salt Form 4A are shown in FIGS. 4-7.

TGA thermograms of phosphate salt Form 4A are shown in FIGS. 4-6.

### Example 7

### DVS of Crystalline 4-Chloro-N-(4-(morpholinomethyl)phenyl)benzamide

### Phosphate Salt Form 4A

A DVS isotherm with a complete sorption and sorption cycle for crystalline phosphate salt Form 4A is shown in FIG. 8A. The DVS kinetic profile is shown in FIG. 8B. A weight gain of up to about 2.2% was observed from 0% to 95% relative humidity, suggesting that the material was hygroscopic. The XRPD of the post-DVS material was the same as that of the initial dry material.

### Example 8

### Solubility of Crystalline 4-Chloro-N-(4-(morpholinomethyl)phenyl)benzamide

### Phosphate Salt Form 4A

The solubility of the crystalline phosphate salt Form 4A and the free base in various solvent systems was determined using HPLC with the instrument parameters shown in Table 7.

TABLE 7
HPLC Solubility Method Parameters.
Instrument | Parameters

| Instrument | Agilent 1260/1290 Infinity | |
|---|---|---|
| Column | Zorbax Eclipse XDB-C18 | |
| | (4.6 × 150 mm, 5 μm) | |
| Column Temp | 45 | °C |
| Wavelengths and | 207, 14 | |
| Bandwidths (nm) | | |
| Mobile Phase A | 0.05% TFA in Water | |
| Mobile Phase B: | 0.05% TFA in ACN | |
| Flow Rate | 0.75 | mL/min |
| Injection Volume | 2 | μL |

| | |
|---|---|
| Needle Wash | ACN:Water 1:1 |
| Diluent | DMSO |

| Time | % A | % B |
|---|---|---|
| 0 | 95 | 5 |
| 13 | 5 | 95 |
| 14 | 5 | 95 |
| 14.1 | 95 | 5 |
| 16 | 95 | 5 |

To determine the solubility of the free base, about 8 mg solids was added to 4 mL solvent and stirred at room temperature to provide a thin off-white slurry. The slurry was sampled at t=0, 2, 4, and 24 hr, syringe-filtered to recover to recover the supernatants, and the diluted sample analyzed using the HPLC method.

To determine the solubility of the crystalline phosphate salt Form 4A, about 40 mg solids was added to 4 mL solvent and stirred at room temperature to form a clear solution. The solution was sampled at t=0 (some solids suspended after about 5 minutes stirring), syringe-filtered to recover the supernatant, and the diluted sample analyzed using the HPLC method.

The results of solvent addition experiments to measure the solubility at room temperature (23° C.) of crystalline phosphate salt Form 4A salt are shown in Table 8.

TABLE 8
Solubility at Room Temperature (23 °C)
of Crystalline Phosphate Salt Form 4A.

| | | RT Solubility |
|---|---|---|
| Solvent | Comments | (mg/mL) |
| Acetone | Did not dissolve in 20 vols. | <50 |
| MeOH | Did not dissolve in 20 vols. | <50 |
| EtOH | Did not dissolve in 20 vols. | <50 |
| IPA | Did not dissolve in 20 vols. | <50 |
| 1-Propanol | Did not dissolve in 20 vols. | <50 |
| MTBE | Did not dissolve in 20 vols. | <50 |
| THF | Did not dissolve in 20 vols. | <50 |
| ACN | Did not dissolve in 20 vols. | <50 |
| DCM | Did not dissolve in 20 vols. | <50 |
| DMF | Did not dissolve in 20 vols. | <50 |
| DMSO | Dissolved in 14 vols. | 71 < S < 74 |
| EtOAc | Did not dissolve in 20 vols. | <50 |
| Heptane | Did not dissolve in 20 vols. | <50 |
| Water | Did not dissolve in 20 vols. | <50 |
| Acetone:Water 90:10 | Did not dissolve in 20 vols. | <50 |
| EtOH:Water 90:10 | Did not dissolve in 20 vols. | <50 |
| DCM:MeOH 1:1 | Did not dissolve in 20 vols. | <50 |
| ACN:Water 1:1 | Did not dissolve in 20 vols. | <50 |
| THF:Water 1:1 | Dissolved in 14 vols. | 71 < S < 74 |
| THF:Water 40:60 | Dissolved in 16 vols. | 63 < S < 67 |
| THF:Water 60:40 | Dissolved in 14.5 vols. | 69 < S < 71 |
| DMSO:Acetone 80:20 | Dissolved in 9.5 vols. | 105 < S < 111 |
| DMSO:Acetone 90:10 | Dissolved in 7.5 vols | 133 < S < 143 |
| DMSO:ACN 80:20 | Dissolved in 14 vols. | 71 < S < 77 |
| DMSO:ACN 90:10 | Dissolved in 9.5 vols. | 105 < S < 111 |
| DMSO:DCM 80:20 | Dissolved in 14 vols. | 71 < S < 77 |
| DMSO:DCM 90:10 | Dissolved in 9 vols. | 111 < S < 125 |
| DMSO:Water 90:10 | Did not dissolve in 20 vols. | <50 |

Solvent addition experiments to measure the solubility at 40° C. of crystalline phosphate salt Form 4A are shown in Table 9. Forty (40) mg of the crystalline phosphate salt Form 4A were weighed into 2 mL vials, and solvent added steps at 0.5-2 vols. at a time, up to 20 vols total.

TABLE 9
Solubility at 40 °C of Crystalline Phosphate Salt Form 4A.

| | | 40 °C |
|---|---|---|
| | | Solubility |
| Solvent | Comments | (mg/mL) |
| Acetone | Did not dissolve in 20 vols. | <50 |
| EtOH | Did not dissolve in 20 vols. | <50 |
| IPA | Did not dissolve in 20 vols. | <50 |
| MTBE | Did not dissolve in 20 vols. | <50 |
| ACN | Did not dissolve in 20 vols. | <50 |
| EtOAc | Did not dissolve in 20 vols. | <50 |
| Heptane | Did not dissolve in 20 vols. | <50 |
| Water | Did not dissolve in 20 vols. | <50 |
| ACN:Water 1:1 | Dissolved in 19 vols. | 53 < S < 56 |
| THF:Water 1:1 | Dissolved in 12.5 vols. | 80 < S < 83 |

The solubility of the freebase and the crystalline phosphate salt Form 4A was assessed in water, FaSSIF, FeSSIF, FaSSGF, pH 2, pH 4, and pH 7.4 media at t=0, 2, 4, and 24 hours using the HPLC solubility method. Solids were slurried in the respective media and sampled at each time point. The results of the solubility assessment are provided in Table 10 and show the crystalline phosphate salt Form 4A salt exhibited enhanced solubility in all media tested compared to the freebase.

TABLE 10
Solubility assessment by HPLC.
HPLC Solubility Assessment (mg/mL)

| Solid | Time (hr) | Water | FaSSIF | FeSSIF | FaSSGF | pH 2 | pH 4 | pH 7.4 |
|---|---|---|---|---|---|---|---|---|
| Free Base | 0 | 0.005 | 0.06 | 0.79 | 9.43 | 6.65 | 0.72 | <0.001 |
| | 2 | 0.004 | 0.07 | 1.11 | 10.68 | 11.16 | 1.26 | <0.001 |
| | 4 | 0.004 | 0.07 | 1.14 | 9.500 | 10.75 | 1.28 | <0.001 |
| | 24 | 0.003 | 0.09 | 1.26 | 10.56 | 10.50 | 1.61 | 0.002 |
| Crystalline | 0 | 8.32 | 0.09 | 6.21 | ≥10 | 7.67 | 7.00 | 0.007 |
| Phosphate | 2 | ≥10 | 0.15 | 6.98 | ≥10 | ≥10 | ≥10 | 0.009 |
| Salt Form 4A | 4 | ≥10 | 0.15 | 7.41 | ≥10 | ≥10 | ≥10 | 0.003 |
| | 24 | ≥10 | 0.17 | 6.25 | ≥10 | ≥10 | ≥10 | 0.002 |

### Example 9

### Stability of Crystalline 4-Chloro-N-(4-(morpholinomethyl)phenyl)benzamide

### Phosphate Salt Form 4A

The stability of crystalline phosphate salt Form 4A, was assessed at 40° C./75% RH for 1 week. About 40 mg of the salt was measured into a 4-mL vial, sealed with Parafilm®, and placed in a 20-mL vial with saturated NaCl solution at about 75% relative humidity. The 20-mL vial was then sealed with Parafilm® and stored in an incubator at 40° C. for 1 week. The sample was then analyzed by XRPD, DSC, TGA, and HPLC.

The crystalline phosphate salt Form 4A was stable at these accelerated test conditions. The results of the XRPD analysis are provided in Table 11.

TABLE 11
Stability Assessment.
Crystalline Phosphate Salt Form 4A Stability Assessment

| Days | XRPD Result | HPLC Purity % |
|---|---|---|
| 0 | Pattern 4A | 98.2% |
| 7 | Pattern 4A | 98.1% |

### Example 10

### Purity of Crystalline 4-Chloro-N-(4-(morpholinomethyl)phenyl)benzamide

### Phosphate Salt Form 4A

The purity of the crystalline phosphate salt Form 4A was determined using HPLC. Three (3) mg of the crystalline phosphate salt Form 4A was dissolved in in 3 mL DMSO and analyzed by HPLC. The instrument and HPLC parameters are provided in Table 12.

TABLE 12
HPLC Purity.
Instrument Parameters

| Instrument | Agilent 1260/1290 Infinity | |
|---|---|---|
| Column | Zorbax Eclipse XDB-C18 | |
| | (4.6 × 150 mm, 5 μm) | |
| Column Temp | 40 | °C |
| Wavelengths and | 265 | nm |
| Bandwidths (nm) | 14 | nm |
| Mobile Phase A | 0.1% TFA in Water | |
| Mobile Phase B | 0.05% TFA in ACN | |
| Flow Rate | 1.0 | mL/min |
| Injection Volume | 5 | μL |

| | |
|---|---|
| Needle Wash | ACN:Water 1:1 |
| Diluent | DMSO |

| Time (min) | % A | % B |
|---|---|---|
| 0 | 90 | 10 |
| 2 | 90 | 10 |
| 17 | 5 | 95 |
| 24 | 5 | 95 |
| 25 | 90 | 10 |
| 32 | 90 | 10 |

The purity of the crystalline phosphate salt Form 4A was determined to be 98%.

### Example 11

### Single Crystal Structure of Crystalline 4-Chloro-N-(4-(morpholinomethyl)phenyl)benzamide

### Phosphate Salt Form 4A

The crystalline phosphate salt Form 4A was recrystallized by placing 12.2 mg of the material into a DSC pan, heating the sample from 30° C. to 190° C. at 10° C./min, and cooling the material from 190° C. to 30° C. at 10° C./min to provide recrystallized crystalline phosphate salt Form 4A.

The best quality crystalline phosphate salt Form 4A crystals were analyzed by single crystal X-ray diffraction (SCXRD) at 100K. These conditions provide an accurate representation of the crystal structure with minimal molecular movement.

The best quality crystalline phosphate salt Form 4A crystals were also analyzed by single crystal X-ray diffraction (SCXRD) at room temperature. SCXRD was performed at room temperature to determine whether the theoretical XRPD crystal structure matches the experimental XRPD of the crystals used in the XRPD analysis.

Single crystals of crystalline phosphate salt Form 4A were grown using anti-solvent vapor diffusion at room temperature and at 40° C. Forty (40) mg of the crystalline phosphate salt Form 4A was placed in 4 mL vial, 14 volumes of solvent were added, and the mixture stirred at RT to dissolve the solids. The sample was placed into a 20 mL vial containing 8 mL anti-solvent and the vial closed and sealed with Parafilm®. After diffusing for 7 days, the off-white solids were analyzed by XRPD, and then dried in a vacuum oven at 40° C.

The solvent/anti-solvent combinations included (1) 1:1 THF:water/THF; (2) 1:1 THF:water/water; (3) 60:40 THF:water/THF; (4) 40:6 THF:water/water; (5) DMSO/acetone; (6) 90:10 DMSO:acetone/acetone; (7) 80:20 DMSO:acetone/acetone; (8) DMSO/CAN; (9) 90:10 DMSO:ACN/ACN; (10) 80:20 DMSO:ACN/ACN; (11) DMSO/DCM; (12) 90:10 DMSO:DCM/DCM; (13) 80:20 DMSO:DCM/DCM; (14) 1:1 ACN:water/water; (15) 1:1 ACN:water/ACN.

The best quality crystals were analyzed by Single Crystal X-ray Diffraction (SCXRD) at 100K and at room temperature. At both temperatures, single crystal structure analysis confirmed that crystalline phosphate salt Form 4A is a mono-salt with a 1:1 molar ratio of phosphoric acid and freebase.

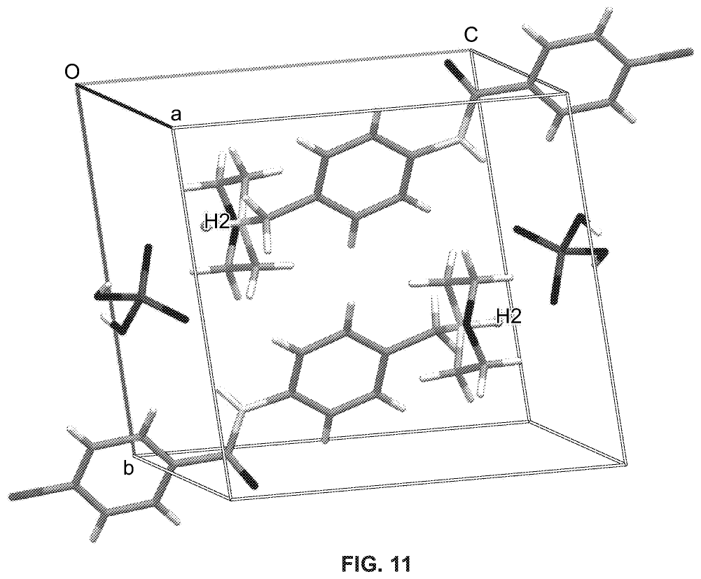

The single crystal lattice parameters and a diagram of the 3-dimensional unit cell for crystalline phosphate salt Form 4A at 100K are provided in Table 13 and in FIG. 11, respectively.

TABLE 13
Single Crystal Structure Analysis of
Crystalline Phosphate Salt Form 4A at 100K.
Single Crystal Structure Analysis at 100K

| | | |
|---|---|---|
| Unit Cell | a = 7.8962(5) Å | α = 83.496(2)° |
| Dimensions | b = 10.4152(7) Å | β = 89.794(2)° |
| | c = 11.4575(7) Å | γ = 86.691(2)° |

| | |
|---|---|
| Crystal System | Triclinic |
| Space Group | P-1 |
| Volume | 934.64(10) Å³ |
| Z | 2 |

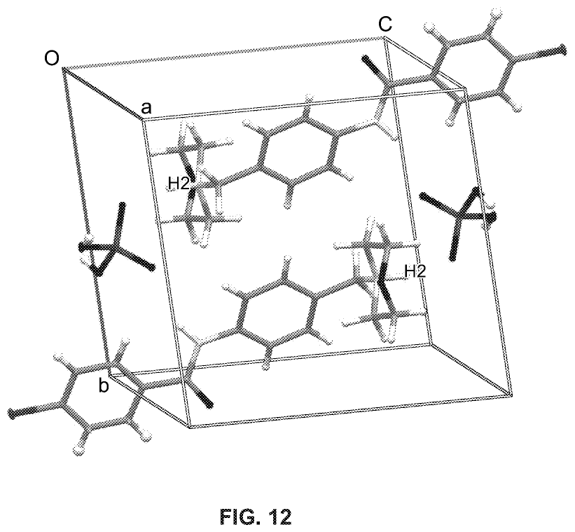

The single crystal lattice parameters and a diagram of the 3-dimensional unit cell for crystalline phosphate salt Form 4A at room temperature (23° C.) are provided in Table 14 and in FIG. 12, respectively.

TABLE 14
Single Crystal Structure Analysis of
Crystalline Phosphate Salt Form 4A at 23 °C
Single Crystal Structure Analysis at 23 °C

| | | |
|---|---|---|
| Unit Cell | a = 7.970(2) Å | α = 83.629(9)° |
| Dimensions | b = 10.642(3) Å | β = 89.888(10)° |
| | c = 11.502(3) Å | γ = 85.054(9)° |

| | |
|---|---|
| Crystal System | Triclinic |
| Space Group | P-1 |
| Volume | 965.90(10) Å³ |
| Z | 2 |

### Example 12

### Single Crystal Analysis of Crystalline Phosphate Salt Form 4A

Single crystal X-ray diffraction studies were carried out on a Bruker D8-Venture 3-circle diffractometer equipped with a Photon III detector and Mo K_{α} radiation (λ=0.7107 Å).

Crystals of the crystalline phosphate salt Form 4A were used as received. A 0.25×0.23× 0.18 mm crystal was mounted on a Cryoloop® with Paratone® oil. Data was collected in a nitrogen gas stream at 100 (2) K using ω scans. The crystal-to-detector distance was 50 mm and the exposure time was 1 or 3 seconds per frame using a scan width of 0.70°. Data collection was 99.9% complete to 25.242° in θ. A total of 27,612 reflections were collected covering the indices, −9≤h≤9, −13≤k≤13, −14≤1≤ 14. Three-thousand eight hundred and sixty (3,860) reflections were found to be symmetry independent, with a Rᵢₙₜ of 0.0306. Indexing and unit cell refinement indicated a triclinic lattice. The space group was P-1. The data was integrated using the Bruker SAINT® Software program and scaled using the SADABS® software program. Solution by direct methods (SHELXT) produced a complete phasing model consistent with the proposed structure.

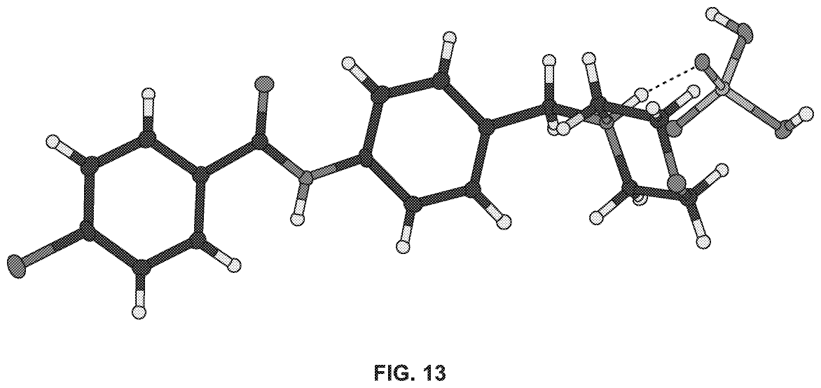

All nonhydrogen atoms were refined anisotropically by full-matrix least-squares (SHELXL-2014). All carbon atoms bonded to hydrogen atoms were placed using a riding model. The positions were constrained relative to the parent atom using the appropriate HFIX command in SHELXL-2014. Crystallographic data are summarized in Table 15 and the structure shown in FIG. 13.

There is one copy of the compound in the asymmetric unit. The chemical formula for the compound is C₁₈ H₁₉CIN₂O₂·H₃PO₄.

TABLE 15
Crystal Data and Structure Refinement.

| Parameter | Value | |
|---|---|---|
| Empirical formula | C₁₈H₂₂ClN₂O₆P | |
| Formula weight | 428.79 g/mol | |
| Temperature | 100K | |
| Wavelength | 0.71073 Å | |
| Crystal system | Triclinic | |
| Space group | P-1 | |
| Unit cell dimensions | a = 7.8962(5) Å | a = 83.496(2)° |
| | b = 10.4152(7) Å | b = 89.794(2)° |
| | c = 11.4575(7) Å | g = 86.691(2)° |

| | |
|---|---|
| Volume | 934.64(10) Å³ |
| Z | 2 |
| Density (calculated) | 1.524 Mg/m³ |
| Absorption coefficient | 0.330 mm⁻¹ |
| F(000) | 448 |
| Crystal size | 0.25 × 0.23 × 0.18 mm³ |
| Theta range for data collection | 2.508° to 26.538° |
| Index ranges | −9 ≤ h ≤ 9, −13 ≤ |
| | k ≤ 13, −14 ≤ l ≤ 14 |
| Reflections collected | 27612 |
| Independent reflections | 3868 [R(int) = 0.0415] |
| Completeness to theta = 25.242° | 99.9% |
| Absorption correction | Semi-empirical from equivalents |
| Max. and min. transmission | 0.4908 and 0.4519 |
| Refinement method | Full-matrix least-squares on F² |
| Data/restraints/parameters | 3868/0/255 |
| Goodness-of-fit on F² | 1.060 |
| Final R indices [I > 2sigma(I)] | R1 = 0.0287, wR2 = 0.0735 |
| R indices (all data) | R1 = 0.0303, wR2 = 0.0751 |
| Largest diff. peak and hole | 0.388 and −0.399 e · Å⁻³ |

### Example 13

### Preparation of Crystalline 4-Chloro-N-(4-(morpholinomethyl)phenyl)benzamide

### Phosphate Salt Form 4C

To prepare crystalline phosphate salt form 4C, 300 mg of the free base was added to 10 vols. solvent (THF:Water (1:1)) to provide an off-white slurry to which a 2.1:1 molar ratio of phosphoric acid was added. The mixture was stirred at 40° C. to provide a clear solution upon counterion addition. The solvent was evaporated for 3 days to provide a thick off-white slurry after 3 days. The material was analyzed by XRPD, vacuum filtered, and dried in a vacuum oven at 40° C. to provide 203.40 mg (52% yield) of the crystalline phosphate salt Form 4C.

The KFT showed a water content of ~7.26%. TGA results showed a ~1.35% weight loss from 30° C. to 150° C. Compared to the TGA weight loss, the significant water content in the KFT result confirmed the hygroscopic nature of the crystalline phosphate salt Form 4C.

### Example 14

### XRPD of Crystalline 4-Chloro-N-(4-(morpholinomethyl)phenyl)benzamide

### Phosphate Salt Form 4C

FIG. 9 shows an XRPD pattern of the crystalline phosphate salt Form 4C. The diffraction peak listing is shown in Table 16.

TABLE 16
Crystalline Phosphate Salt Form 4C XRPD Peak Listing.
Crystalline Phosphate Salt Form 4C Peak Listing

| Angle (°2θ) | Intensity | Relative Intensity |
|---|---|---|
| 6.7 | 40.2 | 6% |
| 7.0 | 68.7 | 11% |
| 11.9 | 15.3 | 2% |
| 13.5 | 113.2 | 18% |
| 13.7 | 167.0 | 27% |
| 14.6 | 14.3 | 2% |
| 15.4 | 49.0 | 8% |
| 15.9 | 98.7 | 16% |
| 16.3 | 17.5 | 3% |
| 16.6 | 68.2 | 11% |
| 17.3 | 213.6 | 34% |
| 18.2 | 80.4 | 13% |
| 18.3 | 65.5 | 11% |
| 19.5 | 172.6 | 28% |
| 19.8 | 623.3 | 100% |
| 20.3 | 72.2 | 12% |
| 20.5 | 168.1 | 27% |
| 21.5 | 50.9 | 8% |
| 22.0 | 14.0 | 2% |
| 22.6 | 190.9 | 31% |
| 23.5 | 76.4 | 12% |
| 24.0 | 119.5 | 19% |
| 24.4 | 110.6 | 18% |
| 25.1 | 388.6 | 62% |
| 26.0 | 85.1 | 14% |
| 26.2 | 112.0 | 18% |
| 26.6 | 92.7 | 15% |
| 27.1 | 87.3 | 14% |
| 27.8 | 36.1 | 6% |
| 28.1 | 73.4 | 12% |
| 28.3 | 68.9 | 11% |
| 29.1 | 85.7 | 14% |
| 29.8 | 47.7 | 8% |
| 30.5 | 81.5 | 13% |
| 31.0 | 21.0 | 3% |
| 31.8 | 48.1 | 8% |
| 32.6 | 21.4 | 3% |
| 33.7 | 14.1 | 2% |
| 34.3 | 32.2 | 5% |
| 34.6 | 49.0 | 8% |
| 35.0 | 38.9 | 6% |
| 35.3 | 28.6 | 5% |
| 35.9 | 2.0 | 0% |
| 36.6 | 15.3 | 2% |
| 37.8 | 34.9 | 6% |
| 38.4 | 26.9 | 4% |
| 39.6 | 14.5 | 2% |

### Example 15

### Characterization of Crystalline 4-Chloro-N-(4-(morpholinomethyl)phenyl)benzamide

### Phosphate Salt Form 4C

A DSC/TGA thermogram overlay for the phosphate salt Form 4C is shown in FIG. 10. The DSC thermogram (lower curve) shows two broad endotherms with onset temperatures at about 34.1° C. and about 153.4° C. The TGA thermogram (upper curve) shows a peak at 172° C. a weight loss of about 1.35% from 30° C. to 150° C. and decomposition at higher temperatures.

Finally, it should be noted that there are alternative ways of implementing the embodiments disclosed herein. Accordingly, the present embodiments are to be considered as illustrative and not restrictive, and the claims are not to be limited to the details given herein but may be modified within the scope and equivalents thereof.

What is claimed is:

1. Crystalline 4-chloro-N-(4-(morpholinomethyl)phenyl)benzamide phosphate salt Form 4A, wherein the crystalline phosphate salt Form 4A is characterized by an XRPD pattern comprising characteristic diffraction peaks at least at 7.9°±0.2°, 13.7°±0.2°, 16.8°±0.2°, 17.8°±0.2°, 18.6°±0.2°, 21.3°±0.2°, 23.5°±0.2°, 23.9°±0.2°, and 26.1°±0.2°, expressed as θ/2θ angles and determined using Cu-Kα radiation.

2. The crystalline phosphate salt of claim 1, wherein the crystalline phosphate salt Form 4A is characterized by an XRPD pattern substantially as shown in FIG. 2.

3. The crystalline phosphate salt of claim 1, wherein the crystalline phosphate salt Form 4A is characterized by an XRPD pattern substantially as shown in FIG. 3.

4. The crystalline phosphate salt of claim 1, wherein the crystalline phosphate salt Form 4A has a differential scanning calorimetry curve characterized by a sharp endotherm with an onset temperature from 219° C. to 230° C.

5. The crystalline phosphate salt of claim 1, wherein the crystalline phosphate salt Form 4A has a differential scanning calorimetry curve characterized by an exothermic peak from 229° C. to 232° C.

6. The crystalline phosphate salt of claim 1, wherein the crystalline phosphate salt Form 4A has a differential scanning calorimetry curve characterized by an exothermic enthalpy from 137 J/g to 147 J/g.

7. The crystalline phosphate salt of claim 1, wherein the crystalline phosphate salt Form 4A has a differential scanning calorimetry curve characterized by a broad endotherm with an onset temperature from 165° C. to 174° C.

8. The crystalline phosphate salt of claim 1, wherein the crystalline phosphate salt Form 4A has a differential scanning calorimetry curve characterized by an endothermic peak from 170° C. to 180° C.

9. The crystalline phosphate salt of claim 1, wherein the crystalline phosphate salt Form 4A has a differential scanning calorimetry curve characterized by an endothermic enthalpy from 1 J/g to 3 J/g.

10. The crystalline phosphate salt of claim 1, wherein the crystalline phosphate salt Form 4A has a thermogravimetric analysis curve characterized by a weight loss of from 0% to 0.25% within a temperature range from 30° C. to 150° C., and with decomposition at higher temperatures.

11. The crystalline phosphate salt of claim 1, wherein the crystalline phosphate salt has a purity greater than 95%.

12. The crystalline phosphate salt of claim 1, wherein the crystalline phosphate salt has a purity greater than 98%.

13. A pharmaceutical composition comprising the crystalline phosphate salt of claim 1 and a pharmaceutically acceptable vehicle.

14. The pharmaceutical composition of claim 13, wherein the pharmaceutical composition is an oral formulation.

15. The pharmaceutical composition of claim 13, wherein the pharmaceutical composition comprises a therapeutically effective amount of the phosphate salt for treating a disease in a patient, wherein the disease is selected from a neurodegenerative disease and a neuromuscular degenerative disease.

16. A method of treating a disease in a patient comprising administering to a patient in need of such treatment a therapeutically effective amount of the crystalline phosphate salt of claim 1, wherein the disease is selected from a neurodegenerative disease and a neuromuscular degenerative disease.

17. A method of treating a disease in a patient comprising administering to a patient in need of such treatment a therapeutically effective amount of the crystalline phosphate salt of claim 1, wherein the disease is selected from amyotrophic lateral sclerosis and Duchenne muscular dystrophy.
